(19) United States
(12) Patent Application Publication
(10) Pub. No.: US 2014/0023179 A1
(43) Pub. Date: Jan. 23, 2014

(54) DETECTION LIMIT CALCULATION DEVICE, RADIATION DETECTION DEVICE, RADIOGRAPHIC IMAGE CAPTURE SYSTEM, COMPUTER-READABLE STORAGE MEDIUM, AND DETECTION LIMIT CALCULATION METHOD

(71) Applicant: FUJIFILM CORPORATION, TOKYO (JP)

(72) Inventor: Yasufumi ODA, Ashigarakami-gun (JP)

(73) Assignee: FUJIFILM CORPORATION, TOKYO (JP)

(21) Appl. No.: 13/947,655

(22) Filed: Jul. 22, 2013

(30) Foreign Application Priority Data
Jul. 23, 2012 (JP) 2012-162565
Jun. 11, 2013 (JP) 2013-123102

(51) Int. Cl. A61B 6/00

(52) U.S. Cl. CPC A61B 6/542
USPC 378/62; 378/91

(57) ABSTRACT

A detection limit calculation device is provided that includes a calculation means that calculates, for image capture of a radiographic image of an imaging subject using an image capture means, a detection limit of a detection means that detects whether irradiation of radiation has started based on a radiation amount of radiation including radiation that has been irradiated and passed through the imaging subject, based on imaging subject data relating to the imaging subject or irradiation data relating to irradiation of radiation or both thereof.

### CROSS-REFERENCE TO RELATED APPLICATION

This application is based on and claims priority under 35 USC 119 from Japanese Patent Application No. 2012-162565 filed on Jul. 23, 2012, and Japanese Patent Application No. 2013-123102 filed on Jun. 11, 2013, the disclosures of which are incorporated by reference herein.

### BACKGROUND

1. Technical Field

The present invention relates to a detection limit calculation device, a radiation detection device, a radiographic image capture system, a computer-readable storage medium, and a detection limit calculation method, and in particular to a detection limit calculation device, a radiation detection device, a radiographic image capture system, a computer-readable storage medium, and a detection limit calculation method related to detection of radiation irradiation start.

2. Related Art

Radiographic image capture apparatuses are known that perform radiographic image capture for the purposes of medical diagnosis. Such radiographic image capture apparatuses detect radiation that has been irradiated from a radiation irradiation device and passed through an investigation subject to capture a radiographic image. Such radiographic image capture apparatuses perform radiographic image capture by storing and reading charges generated according to the irradiated radiation. Generally, such radiographic image capture apparatuses include a sensor portion in which for example a photoelectric conversion element generates charge on irradiation with radiation or illumination with light into which the radiation has been converted, and a switching element that reads the charges generated in the sensor portion.

In such radiographic image capture apparatuses, sometimes appropriate radiographic image capture cannot be performed due to the radiation amount of irradiated radiation. For example, when the amount of irradiated radiation is too low, sometimes a radiographic image cannot be generated. Japanese Patent Application Laid-Open (JP-A) No. 2011-152406 (Patent Document 1) discloses technology wherein an irradiation amount of radiation per single image is calculated, and determination is made as to whether or not the calculated radiation amount is below a minimum irradiation amount required to capture a radiographic image. Moreover, JP-A No. 2008-000595 (Patent Document 2) discloses technology wherein control parameters are determined and an X-ray source is controlled based on irradiation characteristic data using Automatic Exposure Control (AEC) of radiation.

Radiographic image capture apparatuses also exist in which detection of radiation irradiation start (radiographic image capture start) is made based on charges generated in a sensor portion. In such radiographic image capture apparatuses, sometimes radiation irradiation start cannot be detected due to the radiation irradiation amount, or due to changes in the radiation amount with time. For example, specific conditions for the detection of radiation irradiation start are not met when the radiation amount is low, or when the change over time in the radiation amount is small, and radiation irradiation start cannot be detected regardless of the fact that radiation is being irradiated. In such cases, there are concerns of an imaging subject being unnecessarily exposed to radiation since radiographic image data is not acquired even though the imaging subject is exposed to radiation. There are also concerns of time elapsing between the actual start of radiation irradiation and the specific conditions referred to above being satisfied, increasing the radiation exposure amount to the imaging subject.

### SUMMARY

The present invention addresses the above issues, and an object of the present invention is to provide a detection limit calculation device, a radiation detection device, a radiographic image capture system, a computer readable storage medium, and a detection limit calculation method that enable unnecessary radiation exposure to an imaging subject to be suppressed.

A detection limit calculation device of the present invention includes: a calculation unit that calculates, for image capture of a radiographic image of an imaging subject using an image capture unit, a detection limit of a detection unit that detects whether irradiation of radiation has started based on a radiation amount of radiation including radiation that has been irradiated and passed through the imaging subject, based on imaging subject data relating to the imaging subject or irradiation data relating to irradiation of radiation or both thereof.

A radiation detection device of the present invention includes: a detection unit that, for image capture of a radiographic image of an imaging subject with an image capture unit, detects whether irradiation of radiation has started based on a radiation amount of radiation including radiation that has been irradiated and passed through the imaging subject; and the detection limit calculation device of the present invention that calculates the detection limit of the detection unit.

A radiographic image capture system of the present invention includes: a detection unit that, for image capture of a radiographic image of an imaging subject with an image capture unit, detects whether irradiation of radiation has started based on a radiation amount of radiation containing radiation that has been irradiated and passed through the imaging subject; the detection limit calculation device of the present invention that calculates a detection limit of the detection unit; and a control device that controls the image capture unit.

A radiographic image capture system of the present invention includes: a radiographic image capture apparatus that includes a detection unit that detects whether irradiation of radiation has started, and an image capture unit that captures a radiographic image of an imaging subject according to irradiated radiation based on a detection result of the detection unit; and the detection limit calculation device of the present invention that calculates a detection limit of the detection unit.

A radiographic image capture system of the present invention includes: an irradiation device that irradiates radiation; a radiographic image capture apparatus that includes a detection unit that detects whether irradiation of radiation by the irradiation device has started, and an image capture unit that captures a radiographic image of an imaging subject according to irradiated radiation based on a detection result of a detection unit; and the detection limit calculation device of the present invention that calculates the detection limit of the detection unit.

A computer-readable storage medium of the present invention is stored with a detection limit calculation program that causes a computer to function as a calculation unit that calculates, for image capture of a radiographic image of an imaging subject with an image capture unit, a detection limit of a detection unit that detects whether irradiation of radiation has started based on a radiation amount of radiation including radiation that has been irradiated and passed through the imaging subject, based on imaging subject data relating to the imaging subject or irradiation data relating to irradiation of radiation or both thereof

A detection limit calculation method of the present invention includes: calculating, for image capture of a radiographic image of an imaging subject with an image capture unit, a detection limit of a detection unit that detects whether irradiation of radiation has started based on a radiation amount of radiation including radiation that has been irradiated and passed through the imaging subject, based on imaging subject data relating to the imaging subject or irradiation data relating to irradiation of radiation or both thereof.

### BRIEF DESCRIPTION OF THE DRAWINGS

An exemplary embodiment of the present invention will be described in detail based on the following figures, wherein:

FIG. 1 is a schematic configuration diagram illustrating a schematic configuration of an example of a radiographic image capture system according to the present exemplary embodiment;

FIG. 2 is a schematic configuration diagram illustrating a schematic configuration of an example of a radiation irradiation source of the present exemplary embodiment;

FIG. 3 is a schematic diagram illustrating an example of an overall configuration of an electronic cassette according to the present exemplary embodiment;

FIG. 4 is a plan view illustrating an example of a configuration of a radiation detection device according to the present exemplary embodiment;

FIG. 5 is a cross-section of an example of a radiation detection device according to the present exemplary embodiment;

FIG. 6 is a cross-section of an example of a radiation detection device according to the present exemplary embodiment;

FIG. 7 is a schematic configuration diagram illustrating an example of a schematic configuration of a signal detection circuit of a radiographic image capture apparatus according to the present exemplary embodiment;

FIG. 8 is a flow chart illustrating flow in an example of detection limit calculation processing according to the present exemplary embodiment;

FIG. 9 is a graph illustrating a specific example of correspondence relationships between body thickness of an imaging subject and X-ray tube current and X-ray tube voltage of a radiation irradiation source at a detection limit in a normal mode of an electronic cassette according to the present exemplary embodiment;

FIG. 10 is a flow chart illustrating a flow in an example of radiographic image capture processing in an electronic cassette according to the present exemplary embodiment;

FIG. 11 is a flow chart illustrating a flow in another example of detection limit calculation processing of the present exemplary embodiment;

FIG. 12 is a flow chart illustrating an example of flow of processing in a case in which capture of a radiographic image is performed forcibly in a radiographic image capture system of the present exemplary embodiment.

FIG. 13 is a configuration diagram illustrating an example of an overall configuration of an electronic cassette in a case in which radiation irradiation start is detected based for example on charges flowing in common electrode lines;

FIG. 14 is a configuration diagram illustrating another example of an overall configuration of an electronic cassette in a case in which radiation irradiation start is detected based for example on charges flowing in common electrode lines; and

FIG. 15 is a configuration diagram illustrating another example of an overall configuration of an electronic cassette in a case in which radiation irradiation start is detected based for example on charges flowing in common electrode lines.

### DETAILED DESCRIPTION

Explanation follows regarding an example of the present exemplary embodiment, with reference to the drawings.

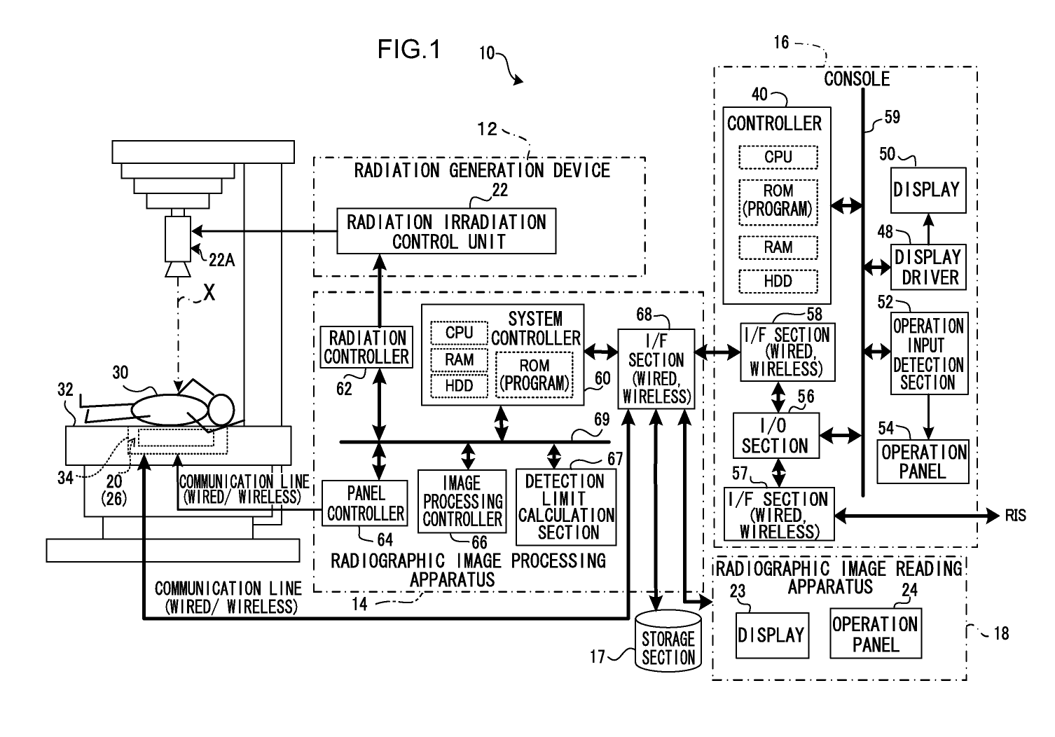

Explanation first follows regarding an overall schematic configuration of a radiographic image capture system equipped with a radiographic image processing apparatus of the present exemplary embodiment. FIG. 1 illustrates a schematic configuration diagram of an overall schematic configuration of an example of a radiographic image capture system of the present exemplary embodiment. In a radiographic image capture system 10 of the present exemplary embodiment, it is possible to capture a still image as well as a video image as a radiographic image. Note that a video image in the present exemplary embodiment means still images displayed successively at high speed so as to give the appearance of a video image, and is generated by performing a process of capturing a still image, converting to an electrical signal, transmitting the electrical signal and reproducing a still image from the transmitted electrical signal repeatedly at high speed. Consequently, this also includes video images referred to as “frame-by- frame” in which, depending on the degree of “high speed”, the same region (part or all) is captured plural times within a predetermined duration and successively reproduced. Moreover, in the radiographic image capture system 10 of the present exemplary embodiment, an electronic cassette 20 itself includes a function to detect irradiation start of radiation (image capture start).

The radiographic image capture system 10 of the present exemplary embodiment includes a function to perform radiographic image capture based on an instruction (image capture menu) input through a console 16 from an external system (for example a Radiology Information System (RIS)), by operation such as by a doctor or radiologist.

Moreover, the radiographic image capture system 10 according to the present exemplary embodiment includes a function for a doctor or radiologist, for example, to read a radiographic image by displaying a captured radiographic image on a display 50 of the console 16 or on a radiographic image reading apparatus 18.

The radiographic image capture system 10 of the present exemplary embodiment includes a radiation generation device 12, a radiographic image processing apparatus 14, the console 16, a storage section 17, the radiographic image reading apparatus 18 and the electronic cassette 20.

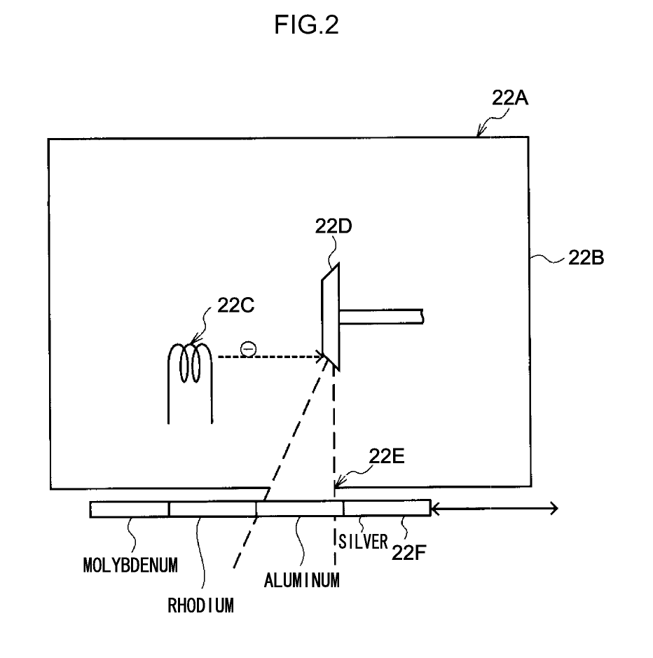

The radiation generation device 12 includes a radiation irradiation control unit 22. The radiation irradiation control unit 22 includes a function to cause radiation X to be irradiated from a radiation irradiation source 22A onto an imaging target site of an investigation subject 30 on an imaging table 32, under control of a radiation controller 62 of the radiographic image processing apparatus 14. FIG. 2 is a schematic configuration diagram illustrating an example of the radiation irradiation source 22A of the present exemplary embodiment.

The radiation irradiation source 22A includes, inside a case 22B, a cathode 22C configured to include a filament, and a target (anode) 22D. Thermions given off by the cathode 22C are accelerated and converged by the potential difference across the cathode and anode, and collide with the target 22D, causing bremsstrahlung to be emitted. Note that in the present exemplary embodiment, plural of the radiation irradiation sources 22A are provided, and different various types of metal are employed for the targets 22D, such as for example tungsten, molybdenum and rhodium. The strength of the generated bremsstrahlung varies depending on the target type.

The radiation X generated by the radiation irradiation source 22A is externally irradiated through a window 22E provided in the case 22B. The window 22E portions are provided with filters 22F that are respectively configured from films of molybdenum, rhodium, aluminum, and silver.

The filters 22F can be moved or changed in the radiation irradiation source 22A of the present exemplary embodiment using a mechanical mechanism (for example guide rails, not shown in the drawings). The characteristics of the radiation X irradiated onto the investigation subject 30 change when the filters 22F are changed.

The radiation X that has passed through the investigation subject 30 reaches the electronic cassette 20 that is held by a holder 34 inside the imaging table 32. The electronic cassette 20 includes functions for generating charge according to the radiation X radiation amount that has passed through the investigation subject 30, and for generating and outputting image data expressing a radiographic image based on the generated charge amount. The electronic cassette 20 of the present exemplary embodiment is configured including a radiation detection device 26. Note that in the present exemplary embodiment “radiation amount” means radiation intensity, for example the radiation irradiated per unit time using a specific X-ray tube voltage and specific X-ray tube current.

In the present exemplary embodiment, image data expressing a radiographic image output by the electronic cassette 20 is input to the console 16 through the radiographic image processing apparatus 14. The console 16 of the present exemplary embodiment employs for example an image capture menu and various information acquired for example from an external system (RIS) through wireless communication (Local Area Network (LAN)), and includes a function to perform control on the radiation generation device 12 and the electronic cassette 20. Moreover, the console 16 of the present exemplary embodiment includes a function to perform transmission and reception of various data including image data of a radiographic image to and from the radiographic image processing apparatus 14, and a function to perform transmission and reception of various data to and from the electronic cassette 20.

The console 16 of the present exemplary embodiment is configured as a server/computer, and is configured including a controller 40, a display driver 48, the display 50, an operation input detection section 52, an operation panel 54, an I/O section 56, an I/F section 57 and an I/F section 58.

The controller 40 includes a function to control the operation of the console 16 overall, and includes a CPU, ROM, RAM and Hard Disk Drive (HDD). The CPU includes a function to control operation of the console 16 overall. The ROM is pre-stored for example with various programs including a control program used by the CPU. The RAM includes a function to temporarily store various data, and the HDD includes a function to store and hold various data.

The display driver 48 includes a function to control display of various data on the display 50. The display 50 of the present exemplary embodiment includes a function to display for example an image capture menu and captured radiographic images. The operation input detection section 52 includes a function to detect an operation state of the operation panel 54. The operation panel 54 is for input of operation instruction related to capturing radiographic images by, for example a doctor or radiologist. The operation panel 54 in the present exemplary embodiment is, for example configured including a touch panel, a touch pen, plural keys and/or a mouse. Note that in cases in which the operation panel 54 is configured as a touch panel then this may be a common configuration to the display 50.

Moreover, the I/O section 56 and the I/F section 58 include functions to perform transmission and reception of various data to and from the radiographic image processing apparatus 14 and the radiation generation device 12, and to perform transmission and reception of various data such as image data to and from the electronic cassette 20, using wireless communication. The I/F section 57 includes a function to perform transmission and reception of various data to and from the RIS.

The controller 40, the display driver 48, the operation input detection section 52, and the I/O section 56 are connected together through a bus 59 such as a system bus or a control bus, so as to enable transmission and reception of data therebetween. The controller 40 accordingly controls display of the various data on the display 50 through the display driver 48, and is also capable of respectively controlling the transmission and reception of various data to and from the radiation generation device 12 and the electronic cassette 20 through the I/F section 58.

The radiographic image processing apparatus 14 of the present exemplary embodiment includes a function to control the radiation generation device 12 and the electronic cassette 20 based on instructions from the console 16, and includes a function to control storage of radiographic images received from the electronic cassette 20 on the storage section 17, and to control display thereof on the display 50 of the console 16 and the radiographic image reading apparatus 18.

The radiographic image processing apparatus 14 according to the present exemplary embodiment is configured including a system controller 60, the radiation controller 62, a panel controller 64, an image processing controller 66, a detection limit calculation section 67, and an I/F section 68.

The system controller 60 includes a function to control the radiographic image processing apparatus 14 overall, and includes a function to control the radiographic image capture system 10. The system controller 60 includes a CPU, ROM, RAM and HDD. The CPU includes a function to control operation of the radiographic image processing apparatus 14 overall and operation of the radiographic image capture system 10. The ROM is pre-stored for example with various programs including a control program used by the CPU. The RAM includes a function to temporarily store various data, and the HDD includes a function to store and hold various data. The radiation controller 62 includes a function to control the radiation irradiation control unit 22 of the radiation generation device 12 based on for example instructions of the console 16. The panel controller 64 includes a function to control the electronic cassette 20 based on for example instructions of the console 16. The image processing controller 66 includes a function to subject radiographic images to various types of image processing. The detection limit calculation section 67 includes a function to calculate a detection limit in the electronic cassette 20 for start of irradiation of radiation X from the radiation generation device 12 (described in detail later).

The system controller 60, the radiation controller 62, the panel controller 64, the image processing controller 66 and the detection limit calculation section 67 are connected together through a bus 69 such as a system bus or a control bus, to enable for example sending and receiving of data therebetween.

The storage section 17 of the present exemplary embodiment includes a function to store captured radiographic images and data related to the captured radiographic images. An example of the storage section 17 is a HDD.

Moreover, the radiographic image reading apparatus 18 of the present exemplary embodiment is an apparatus that includes a function for a reader to read captured radiographic images. There are no particular limitations thereto, however examples include what is referred to as a radiogram interpretation viewer, and a console. The radiographic image reading apparatus 18 of the present exemplary embodiment is configured by a personal computer, and similarly to the console 16 and the radiographic image processing apparatus 14, is configured including a CPU, ROM, RAM, a HDD, a display driver, a display 23, an operation input section, an operation panel 24, an I/O section and an I/F section. Note that in FIG. 1, in order to avoid making the illustration too complicated, only the display 23 and the operation panel 24 are illustrated out of these configuration items, and the other items are omitted from illustration.

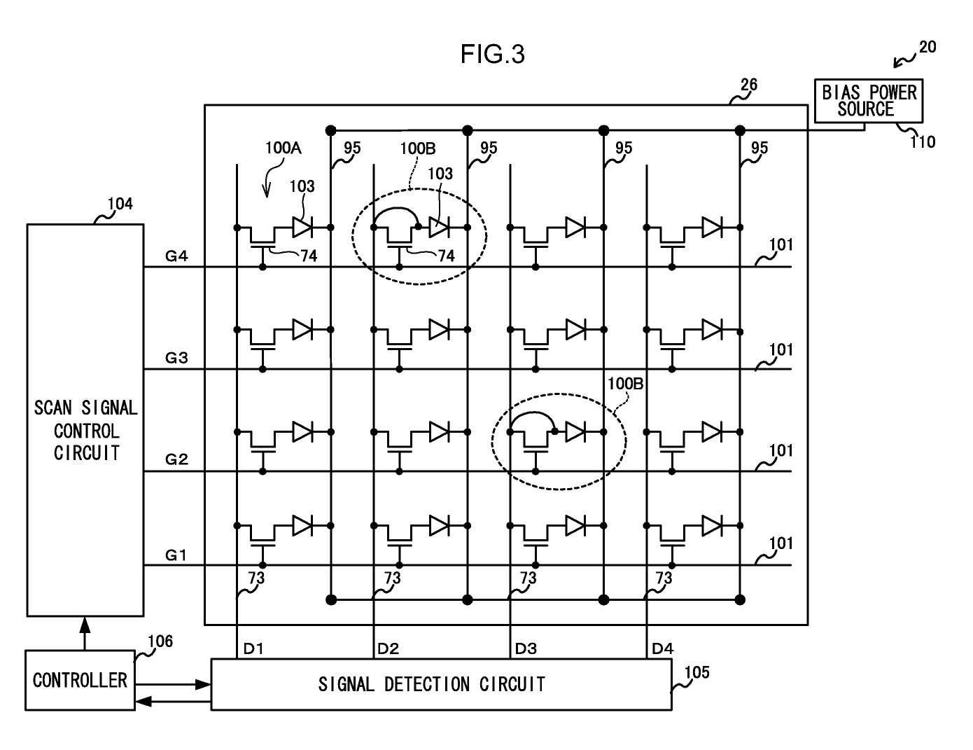

Explanation next follows regarding a schematic configuration of the electronic cassette 20 of the present exemplary embodiment. FIG. 3 illustrates a schematic configuration diagram of an example of the electronic cassette 20 of the present exemplary embodiment. In the present exemplary embodiment, explanation is given regarding a case in which the present invention is applied to an indirect conversion type radiation detection device 26 in which radiation such as X-rays is first converted into light, and then the converted light is converted into charges. In the present exemplary embodiment, the electronic cassette 20 is configured including the indirect conversion type radiation detection device 26. Note that in FIG. 3, the scintillator that converts radiation into light is omitted from illustration.

Plural pixels 100 are disposed in a matrix formation in the radiation detection device 26. Each of the pixels 100 includes: a sensor portion 103 that receives light, generates charge and accumulates the generated charge and a TFT switch 74 that is a switching element that reads charge accumulated in the sensor portion 103. In the present exemplary embodiment, the sensor portions 103 generate charge by illumination with light converted by the scintillator.

Plural of the pixels 100 are disposed in a matrix formation along one direction (a gate line direction in FIG. 3) and a direction intersecting with the gate line direction (a signal line direction in FIG. 3). The array of the pixels 100 is simplified in the illustration of FIG. 3. In reality there are, for example, 1024×1024 individual pixels 100 disposed along the gate line direction and along the signal line direction.

In the present exemplary embodiment, pixels 100A employed in radiographic image capture and pixels 100B employed in radiation detection are predetermined in the plural pixels 100. In FIG. 3, the radiation detection pixels 100B are surrounded by broken lines. The radiographic image capture pixels 100A are employed to detect radiation X and to generate images representing the radiation X. The radiation detection pixels 100B are pixels employed to detect the radiation X in order to detect for example the irradiation start of the radiation X, and are pixels that output charge irrespective of the ON/OFF state of the TFT switches 74, even during charge accumulation periods (described in detail later).

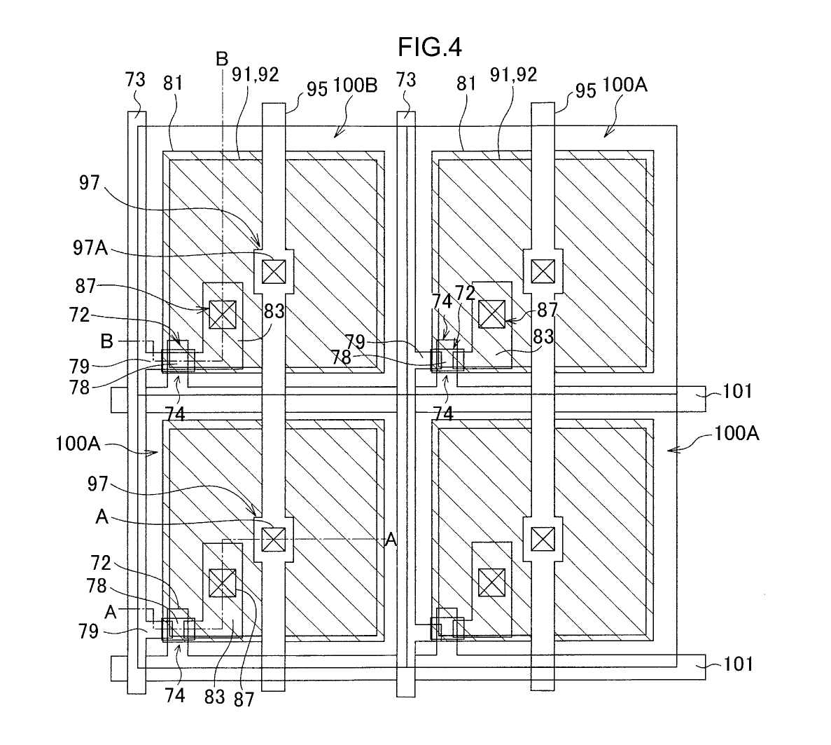

Moreover, in the radiation detection device 26, plural gate lines 101 for switching the TFT switches 74 ON/OFF, and plural signal lines 73 for reading charge accumulated in the sensor portions 103 are provided on a substrate 71 so as to intersect with each other (see FIG. 4). In the present exemplary embodiment there is one each of the signal lines 73 provided for each of the pixel rows in the one direction, and there is one each of the gate lines 101 provided for each of the pixel rows in the intersecting direction, so for example in a case in which there are 1024×1024 of the pixels 100 disposed in the gate line direction and the signal line direction, there are 1024 each of the signal lines 73 and the gate lines 101 provided.

Moreover, in the radiation detection device 26, there are common electrode lines 95 provided parallel to each of the signal lines 73. The common electrode lines 95 have one end and another end connected in parallel, with one end connected to a bias power source 110 that supplies a specific bias voltage. The sensor portions 103 are connected to the common electrode lines 95 and are applied with the bias voltage through the common electrode lines 95.

Scan signals flow in the gate lines 101 for switching each of the TFT switches 74. Each of the TFT switches 74 is accordingly switched by scan signals flowing in each of the gate lines 101.

Electrical signals flow in the signal lines 73 according to the charge accumulated in each of the pixels 100 and according to the switching state of the TFT switches 74 of each of the pixels 100. More specifically, electrical signals corresponding to accumulated charge amounts are caused to flow in each of the signal lines 73 by the TFT switches 74 of the pixels 100 connected to the corresponding signal lines 73 being switched ON.

A signal detection circuit 105 is connected to each of the signal lines 73 and detects electrical signals that have flowed out into each of the signal lines 73. Moreover, a scan signal control circuit 104 is connected to each of the gate lines 101 and outputs scan signals to each of the gate lines 101 for switching the TFT switches 74 ON/OFF. Simplification is made in FIG. 3 to show a single signal detection circuit 105 and a single scan signal control circuit 104, however there are for example plural of the signal detection circuits 105 and the scan signal control circuits 104 provided, connected at one circuit per a specific number (for example 256 lines) of the signal lines 73 and the gate lines 101. For example, in a case in which there are 1024 lines each of the signal lines 73 and the gate lines 101 provided, four of the scan signal control circuits 104 are provided, each connected to 256 of the gate lines 101, and four of the signal detection circuits 105 are provided, each connected to 256 of the signal lines 73.

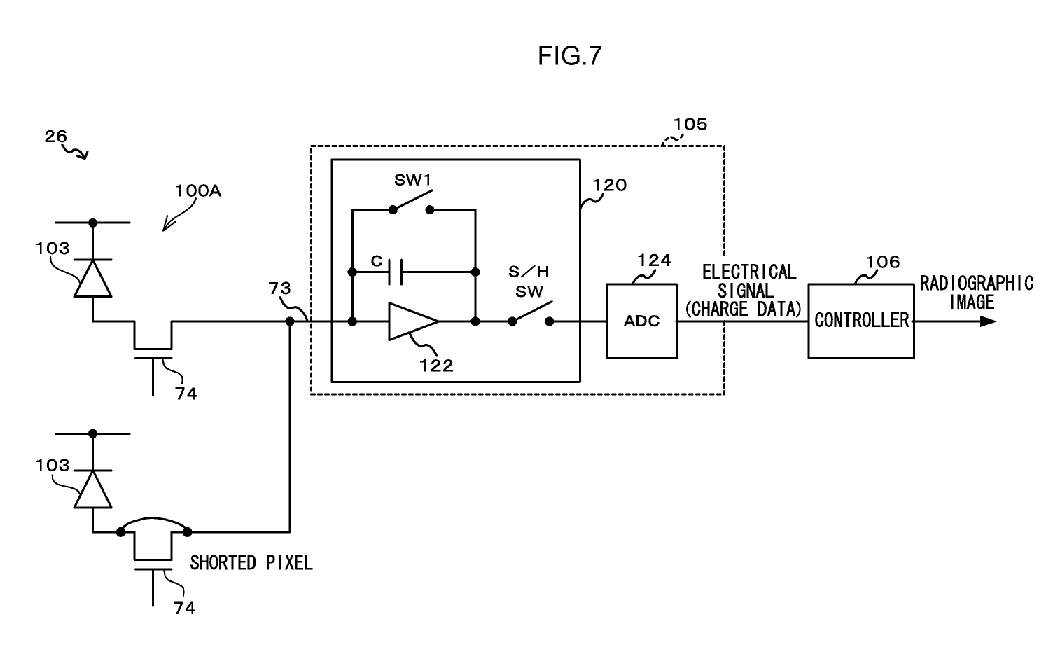

An amplification circuit (see FIG. 7) that amplifies input electrical signals is built into the signal detection circuit 105 for each of the signal lines 73. In the signal detection circuit 105, electrical signals input by each of the signal lines 73 are amplified by the amplification circuit and converted into digital signals by an analogue-digital converter (ADC) (described in detail later).

A controller 106 is connected to the signal detection circuit 105 and the scan signal control circuit 104. The controller 106 performs specific processing, for example noise removal, on the digital signals converted in the signal detection circuit 105 and outputs a control signal to the signal detection circuit 105 to indicate timings for signal detection, and outputs a control signal to the scan signal control circuit 104 to indicate timings for outputting scan signals.

The controller 106 of the present exemplary embodiment is configured by a microcomputer and is provided with a Central Processing Unit (CPU), ROM and RAM, and a non-volatile storage section configured by for example flash memory. The controller 106 performs control to capture radiographic images by using the CPU to execute a program stored in the ROM. The controller 106 also performs processing (interpolation processing) to interpolate image data for each of the radiation detection pixels 100B on image data to which the above specific processing has been performed, so as to generate an image expressing irradiated radiation X. Namely, the controller 106 generates an image expressing the irradiated radiation X by interpolating image data for each of the radiation detection pixels 100B based on the image data that has been subjected to the above specific processing.

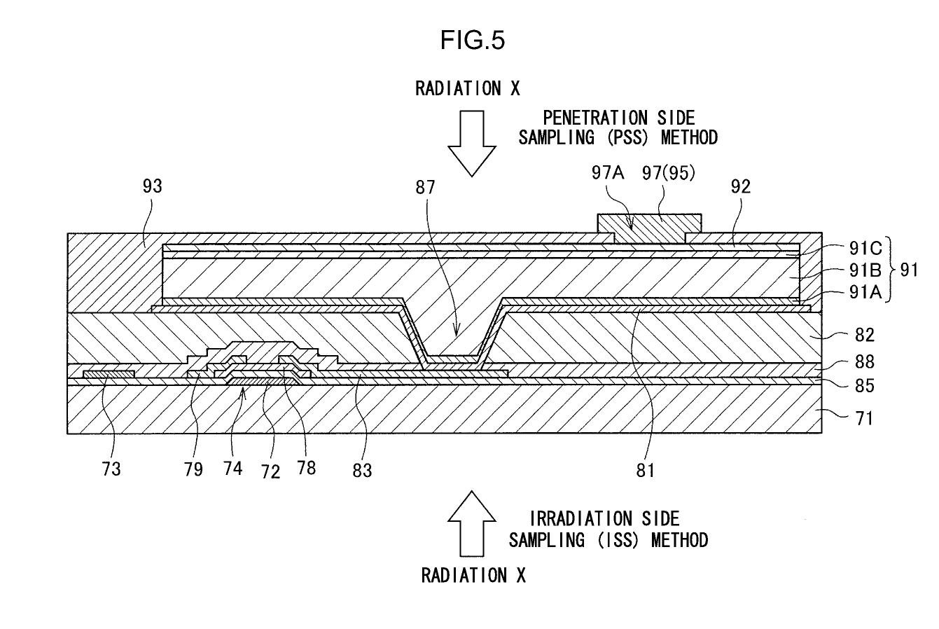

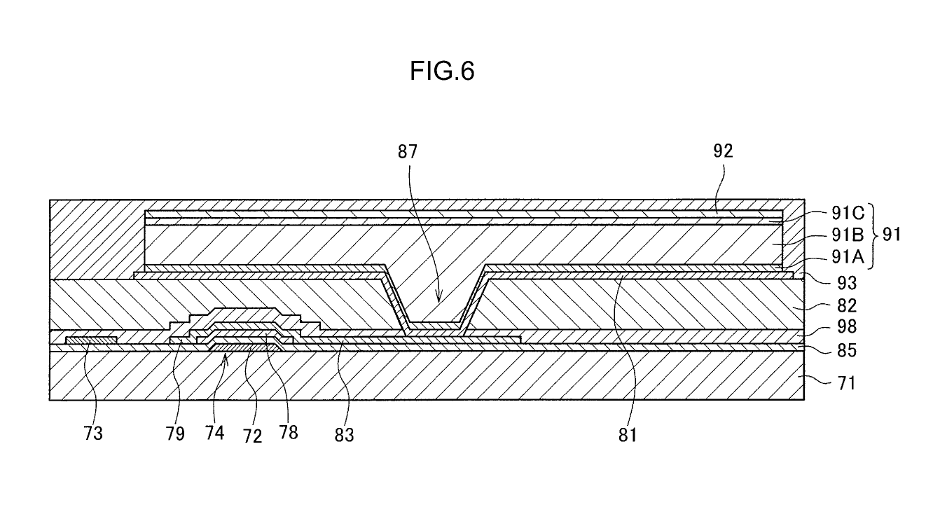

FIG. 4 is a plan view illustrating structure of the indirect conversion type radiation detection device 26 according to the present exemplary embodiment, FIG. 5 illustrates a cross-section taken on line A-A of the radiographic image capture pixel 100A of FIG. 4, and FIG. 6 is a cross-section taken on line B-B of the radiation detection pixel 100B of FIG. 4.

As illustrated in FIG. 5, the pixels 100A of the radiation detection device 26 include the gate line 101 (see FIG. 4) and a gate electrode 72 formed on the insulating substrate 71, such as of non-alkali glass, with the gate line 101 and the gate electrode 72 connected together (see FIG. 4). The wiring layer in which the gate lines 101 and the gate electrodes 72 are formed (this wiring layer is referred to below as the “first signal wiring layer”) is formed using Al or Cu, or a stacked layer film of mainly Al or Cu, however there is no limitation thereto.

An insulation film 85 is formed on one face on the first signal wiring layer, and locations thereof above the gate electrodes 72 act as gate insulation films in the TFT switches 74. The insulation film 85 is, for example, formed from SiN_{X} using Chemical Vapor Deposition (CVD) film forming.

A semiconductor active layer 78 is formed with an island shape on the insulation film 85 above the gate electrode 72. The semiconductor active layer 78 is a channel portion of the TFT switch 74 and is, for example, formed from an amorphous silicon film.

A source electrode 79 and a drain electrode 83 are formed in a layer above. The wiring layer in which the source electrode 79 and the drain electrode 83 are formed also has the signal line 73 formed therein, as well as the source electrode 79 and the drain electrode 83. The source electrode 79 is connected to the signal line 73 (see FIG. 4). The wiring layer in which the source electrode 79, the drain electrode 83 and the signal line 73 are formed (this wiring layer is referred to below as the “second signal wiring layer”) is formed from Al or Cu, or a stacked layer film mainly composed of Al or Cu, however there is no limitation thereto. An impurity doped semiconductor layer (not shown in the drawings) formed for example from impurity doped amorphous silicon is formed between the semiconductor active layer 78 and both the source electrode 79 and the drain electrode 83. Each of the TFT switches 74 employed for switching is configured with such a configuration. Note that the TFT switches 74 may be configured with the source electrode 79 and the drain electrode 83 interchanged according to the polarity of the charge collected and accumulated by lower electrodes 81, described later.

A TFT protection film layer 98 is formed covering the second signal wiring layer over substantially the whole surface (substantially the entire region) of the region where the pixels 100 are provided on the substrate 71, to protect the TFT switches 74 and the signal lines 73. The TFT protection film layer 98 is formed, for example, from a material such as SiN_{X} by, for example, CVD film forming.

A coated intermediate insulation layer 82 is formed on the TFT protection film layer 98. The intermediate insulation layer 82 is formed from a low permittivity (specific permittivity εr=2 to 4) photosensitive organic material (examples of such materials include positive working photosensitive acrylic resin materials with a base polymer formed by copolymerizing methacrylic acid and glycidyl methacrylate, mixed with a naphthoquinone diazide positive working photosensitive agent) at a film thickness of 1 to 4 μm.

In the radiation detection device 26 according to the present exemplary embodiment, inter-metal capacitance between metal disposed in the layers above the intermediate insulation layer 82 and below the intermediate insulation layer 82 is suppressed to a small capacitance by the intermediate insulation layer 82. Generally such materials also function as a flattening film, exhibiting an effect of flattening out steps in the layers below. In the radiation detection device 26 of the present exemplary embodiment, contact holes 87 are formed at positions where the intermediate insulation layer 82 and the TFT protection film layer 98 face towards the drain electrodes 83.

A lower electrode 81 of each of the sensor portions 103 is formed on the intermediate insulation layer 82 so as to cover the pixel region while also filling the contact hole 87. The lower electrode 81 is connected to the drain electrode 83 of the TFT switch 74. In cases in which the thickness of a semiconductor layer 91, described later, is about 1 μm there are substantially no limitations to the material of the lower electrode 81, as long as it is an electrically conductive material. The lower electrode 81 may therefore be formed using a conductive metal such as an Al material or ITO.

However, there is insufficient light absorption in the semiconductor layer 91 when the film thickness of the semiconductor layer 91 is thin (about 0.2 to 0.5 μm). An alloy or layered film with a main component of a light blocking metal is accordingly preferably employed for the lower electrode 81 in such cases in order to prevent an increase in leak current occurring due to light illumination onto the TFT switch 74.

The semiconductor layer 91 is formed on the lower electrode 81 and functions as a photodiode. In the present exemplary embodiment, a photodiode of PIN structure stacked with an n+ layer, an i layer and a p+ layer (n+ amorphous silicon, amorphous silicon, p+ amorphous silicon) is employed as the semiconductor layer 91, with an n+ layer 21A, an i layer 21B and a p+ layer 21C stacked in this sequence from the bottom layer. The i layer 21B generates charges (pairs of free electrons and free holes) on illumination with light. The n+ layer 21A and the p+ layer 21C function as contact layers and electrically connect the i layer 21B to the lower electrodes 81 and upper electrodes 92, described later.

The upper electrodes 92 are respectively formed separately on each of the semiconductor layers 91. A material with high light-transparency such as ITO or Indium Zinc Oxide (IZO) is for example employed for the upper electrodes 92. In the radiation detection device 26 of the present exemplary embodiment the sensor portions 103 are each configured including the upper electrode 92, the semiconductor layer 91 and the lower electrode 81.

A coated intermediate insulation layer 93 is formed over the intermediate insulation layer 82, the semiconductor layer 91 and the upper electrodes 92, and is formed so as to cover each of the semiconductor layer 91 with openings 97A formed in portions corresponding to above the upper electrodes 92.

The common electrode lines 95 are formed on the intermediate insulation layer 93 from Al or Cu, or from stacked layer films of an alloy of mainly Al or Cu. The common electrode lines 95 are formed in the vicinity of the openings 97A with contact pads 97 that are electrically connected to the upper electrodes 92 through the openings 97A of the intermediate insulation layer 93.

However, as illustrated in FIG. 6, in the radiation detection pixels 100B of the radiation detection device 26, the TFT switches 74 are formed such that the source electrode 79 and the drain electrode 83 are in contact with each other. Namely, in the pixels 100B, the sources and drains of the TFT switches 74 are shorted. Thus in the pixels 100B, charges collected by the lower electrodes 81 flow out to the signal lines 73 irrespective of the switching state of the TFT switches 74.

In the radiation detection device 26 formed in this manner, a further protection layer is formed from an insulating material with low light absorptivity as required, and then the scintillator that serves as a radiation conversion layer is stuck to the surface thereof using a bonding resin with low light absorptivity. Moreover, the scintillator may be formed using a vacuum deposition method. As a scintillator, preferably a scintillator is employed that generates fluorescence having a comparatively wide wavelength region, so as to enable light to be emitted in a wavelength region capable of being absorbed. Examples of materials for such scintillators include CsI: Na, CaWO₄, YTaO₄: Nb, BaFX: Eu (wherein X is Br or Cl), or LaOBr: Tm, and GOS. Specifically, in cases in which image capture is performed employing X-rays as the radiation X, preferably cesium iodide (CsI) is included, and CsI: Tl (thallium doped cesium iodide) or CsI: Na, that have emission spectra of 400 nm to 700 nm when irradiated with X-rays are particularly preferably employed. The emission peak wavelength in the visible light region of CsI: Tl is 565 nm. In cases in which a scintillator containing CsI is employed as the scintillator, preferably an oblong shaped columnar crystal structure is fowled using a vacuum deposition method.

As illustrated in FIG. 5, light is emitted with higher intensity at the upper face in FIG. 5 of the scintillator provided on the semiconductor layer 91 when the radiation detection device 26 is irradiated with radiation X from the side where the semiconductor layer 91 is formed, and radiographic images are read by the TFT substrate provided on the back face side with respect to the radiation X incident face, in what is referred to as a Penetration Side Sampling (PSS) method. However, radiation X that has passed through the TFT substrate is incident to the scintillator and light is emitted with higher intensity from the TFT substrate side of the scintillator in cases in which radiation X is irradiated from the TFT substrate side and radiographic images are read by the TFT substrate provided on the front face side with respect to the radiation X incident face, in what is referred to as an Irradiation Side Sampling (ISS) method. Each of the sensor portions 103 of each of the pixels 100 provided to the TFT substrate generates charges due to the light generated by the scintillator. The radiation detection device 26 therefore gives a higher resolution of captured radiographic images in cases in which an ISS method is employed than in cases in which a PSS method is employed, since the most intense light emission position of the scintillator is closer to the TFT substrate.

Note that the radiation detection device 26 is not limited to that illustrated in FIG. 4 to FIG. 6, and various modifications are possible. For example, in cases in which a PSS method is employed, due to there being only a low possibility of radiation X arriving, a combination of another image pickup device such as a Complementary Metal-Oxide Semiconductor (CMOS) image sensor that has a low durability to radiation X and TFTs may be employed instead of the above configuration. Moreover, configuration may be made so as to replace the radiation detection device 26 with a Charge Coupled Device (CCD) image sensor that transmits charge while shifting by a shift pulse that is equivalent to the scan signal for TFTs.

Moreover, for example, a flexible substrate may be employed. An ultra-thin glass substrate produced by recently developed float technology may be applied as a substrate for such a flexible substrate in order to improve the transmissivity to radiation X. Examples of ultra-thin glass that may be applied in such cases include, for example, that described in “Asahi Glass Company (AGC) Develops Worlds Thinnest Sheet Float Glass at Just 0.1 MM”, Internet <URL:http://vvww.agc.com/news/2011/0516.pdf>(online search Aug. 20, 2011).

Explanation next follows regarding a schematic configuration of a signal detection circuit 105 of the present exemplary embodiment. FIG. 7 is a schematic configuration diagram illustrating an example of the signal detection circuit 105 of the present exemplary embodiment. The signal detection circuit 105 of the present exemplary embodiment is configured including amplification circuits 120 and an analogue-to-digital (ADC) converter 124. Although omitted from illustration in FIG. 7, the amplification circuits 120 are provided for each signal line 73. Namely, the signal detection circuit 105 is configured including the same plural number of amplification circuits 120 as the number of signal lines 73 of the radiation detection device 26.

The amplification circuits 120 are configured as charge amplification circuits and are configured including an amplifier 122 such as an operational amplifier, a condenser C connected in parallel to the amplifier 122, and a switch SW1 that is connected in parallel to the amplifier 122 and is employed in charge resetting. Note that the amplification circuits 120 in the present exemplary embodiment are configured with variable gain (amplification ratio) according to the sensitivity during radiographic image capture.

In the amplification circuits 120, charges (electrical signals) are read by the TFT switches 74 of the pixels 100 whose charge reset switch SW1 is in the OFF state, and the charges read by the TFT switches 74 are accumulated in the condensers C, such that the voltage value output from the amplifier 122 is increased according to the accumulated charge amount.

Moreover, the controller 106 applies a charge reset signal to the charge reset switch SW1 to control to ON/OFF switch the charge reset switch SW1. Note that when the charge reset switch SW1 is in the ON state, the input side and the output side of the amplifier 122 are shorted, and so charge of the condensers C is discharged.

The ADC 124 has a function to convert an electrical signal that is an analogue signal input from the amplification circuits 120 in the ON state of a sample and hold (S/H) switch SW into a digital signal. The ADC 124 sequentially outputs to the controller 106 electrical signals that have been converted into digital signals.

Note that the ADC 124 in the present exemplary embodiment is input with the electrical signal output from all the amplification circuits 120 provided to the signal detection circuit 105. Namely, the signal detection circuit 105 of the present exemplary embodiment is provided with a single ADC 124 irrespective of the number of the amplification circuits 120 (the signal lines 73).

In the present exemplary embodiment, configuration is made such that detection related to irradiation of the radiation X is performed without requiring an external control signal (for example from the radiographic image processing apparatus 14). In the present exemplary embodiment, the electrical signals (charge data) of the signal lines 73 that are connected to the radiation detection pixels 100B (at least one of D2 or D3 in FIG. 3, say D2) are detected by the amplification circuits 120 of the signal detection circuit 105 and converted into digital signals. The control section 106 then compares the value of the rise (the amount of change per unit time) in the digital signal converted by the signal detection circuit 105 against a predetermined detection specific value and, detects whether or not radiation X has been irradiated depending on whether or not the digital signal value is the specific value or greater. Note that detection as to whether or not radiation X has been irradiated is not limited thereto. For example, configuration may be made such that detection is performed by comparing the digital signal against a predetermined detection threshold value and detecting whether or not radiation X has been irradiated by whether or not the digital signal is the threshold value or greater, or configuration may be made such that detection is based on preset conditions such as a number of times that the digital signals is the specific value or greater, or the number of detection times etc.

Note that “detection” of an electrical signal in the present exemplary embodiment refers to electrical signal sampling.

Explanation next follows regarding a flow of operation during radiographic image capture using the electronic cassette 20 configured as described above. In the radiographic image capture system 10 of the present exemplary embodiment, the electronic cassette 20 itself detects the irradiation start of radiation X, and when irradiation start is detected, the electronic cassette 20 performs radiographic image capture by accumulating charge according to the radiation amount of the radiation X that has been irradiated (arrived), reading the accumulated charge and generating a radiographic image. The electronic cassette 20 detects irradiation start of the radiation X based on an electrical signal (charge data) output from the radiation detection pixels 100B. In such cases, sometimes irradiation start cannot be detected due to the radiation amount of the radiation X irradiated onto the electronic cassette 20 (the radiation detection device 26). For example, since in the electronic cassette 20 the arriving irradiated radiation X has passed through the investigation subject 30, the radiation amount that arrives at the electronic cassette 20 is reduced by passing through the investigation subject 30, and sometimes does not reach a limit at which irradiation start detection is possible. In such cases, radiographic images are not generated even though the investigation subject 30 is being exposed to the radiation X. The investigation subject 30 is accordingly subjected to unnecessary radiation exposure.

In the present exemplary embodiment, as a limit to the radiation amount arriving at the electronic cassette 20 that enables detection of the radiation X irradiation start, a limit of the irradiation conditions such as the radiation amount of radiation X irradiated from the radiation generation device 12 and a limit of imaging subject conditions such as the thickness of the imaging subject of the investigation subject 30 are called the “detection limit”. An accumulated charge amount (necessary sensitivity) per unit time of the electronic cassette 20 is determined based on imaging conditions including irradiation conditions and based on imaging subject conditions. The detection sensitivity of the electronic cassette 20 is determined based on this accumulated charge amount. In the present exemplary embodiment “detection sensitivity” means the ability to capture radiation X, and more specifically is an indicator that expresses the ability to capture an appropriate radiographic image for radiation amounts of radiation X. In the electronic cassette 20, the higher the detection sensitivity the smaller the radiation amount with which the electronic cassette 20 is capable of capturing an appropriate radiographic image. The detection limit differs according to the detection sensitivity of the electronic cassette 20. Note that in the present exemplary embodiment, the sensitivity to capture an appropriate radiographic image is employed as the detection sensitivity for detecting radiation X irradiation start.

Accordingly, in the radiographic image capture system 10 of the present exemplary embodiment, due to calculating the detection limit of the electronic cassette 20 during radiographic image capture, unnecessary exposure of the investigation subject 30 is suppressed.

Explanation follows regarding calculation of the detection limit. Note that in the present exemplary embodiment, explanation follows regarding a case in which calculation of the detection limit is performed in the radiographic image processing apparatus 14, however there is no limitation thereto.

In the present exemplary embodiment, the detection limit calculation section 67 of the radiographic image processing apparatus 14 calculates the detection limit using imaging subject data related to the investigation subject 30 or irradiation data related to radiation X irradiation or both. In the present exemplary embodiment “imaging subject data” refers to data about the side through which the radiation X irradiated from the radiation generation device 12 (the radiation irradiation source 22A) will pass through (be absorbed). Specific examples thereof include such factors as the imaging subject site of the investigation subject 30, the body thickness (referred to below as imaging subject thickness), size and shape of the imaging subject site, the height, weight, age and gender of the investigation subject 30, however there is no limitation thereto.

Note that since there is a large influence from body thickness, preferably the body thickness or the height and weight for deriving the body thickness, are contained in the imaging subject data. Moreover, in the present exemplary embodiment “irradiation data” means data on the side radiation X is irradiated, such as the irradiation conditions during radiation X irradiation from the radiation generation device 12 (the radiation irradiation source 22A). Specific examples thereof include such factors as the mAs value, the X-ray tube voltage (kV) and the X-ray tube current (mA) of the radiation irradiation source 22A, the type of the target 22D, the type of the filter 22F, the irradiation duration, and the separation distance between the radiation irradiation source 22A and the investigation subject 30, however there is no limitation thereto.

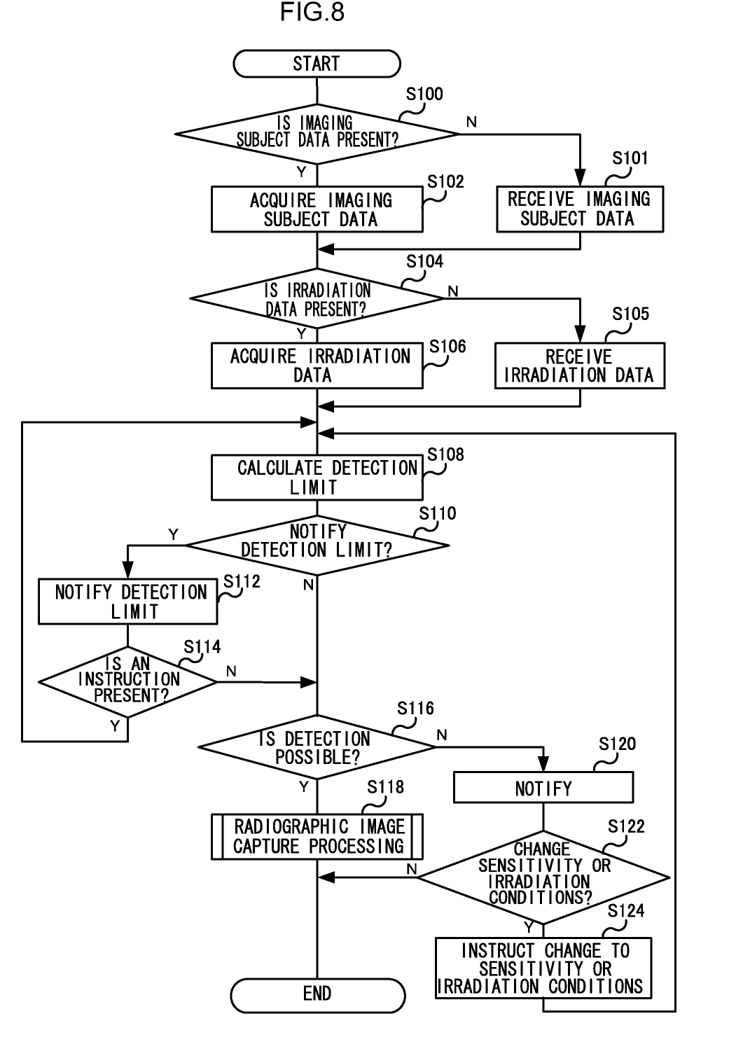

FIG. 8 is a flow chart illustrating a flow of an example of detection limit calculation processing. The detection limit calculation processing illustrated in FIG. 8 is executed by the system controller 60 when the radiographic image processing apparatus 14 has received an instruction to capture a radiographic image.

At step S100 determination is made as to whether or not there is imaging subject data present. The imaging subject data is, for example, sometimes contained in the image capture menu received from the console 16, and is sometimes stored in advance in the storage section 17 or a storage section (not illustrated in the drawings) inside the radiographic image processing apparatus 14. This is searched for in the present exemplary embodiment, and the presence or absence of imaging subject data determined. Note that when there is no imaging subject data, negative determination is made and processing proceeds to step S101. At step S101, after the imaging subject data specified by a user has been received, processing proceeds to step S104. Note that preferably notification to prompt a user to specify imaging subject data is made in such cases. However, affirmative determination is made in cases in which the imaging subject data is present, and then processing proceeds to step S104 after the imaging subject data has been acquired at step S102.

At the next step S104, determination is made as to whether or not irradiation data is present. The irradiation data is, similarly to the imaging subject data, for example contained in the image capture menu received from the console 16, or stored in advance in the storage section 17 or a storage section (not illustrated in the drawings) inside the radiographic image processing apparatus 14. This is searched for in the present exemplary embodiment, and the presence or absence of irradiation data determined. When there is no imaging subject data present, negative determination is made and processing proceeds to step S105. At step S105, after the irradiation data specified by a user has been received, processing proceeds to step S108. Note that preferably notification to prompt a user to specify irradiation data is made in such cases. However, affirmative determination is made in cases in which the irradiation data is present, and then processing proceeds to step S108 after the irradiation data has been acquired at step S106.

At step S108, the detection limit is calculated by the detection limit calculation section 67 based on the imaging subject data or the irradiation data or both. In the present exemplary embodiment, at least one of corresponding relationships (a table) between imaging subject data and detection limit, corresponding relationships between irradiation data and detection limit, and correspondence relationships between imaging subject data and irradiation data and detection limit are stored in advance in a storage section (not illustrated in the drawings) of the radiographic image processing apparatus 14 or the storage section 17.

Moreover, in the electronic cassette 20 of the present exemplary embodiment, the detection limit is calculated based on the detection sensitivity (mode). As described above, the detection limit differs according to the detection sensitivity of the electronic cassette 20. The electronic cassette 20 of the present exemplary embodiment has, as detection sensitivities, a normal sensitivity mode and a high sensitivity mode. In the electronic cassette 20 the normal sensitivity mode is initially set, and image capture is performed using the normal sensitivity mode unless there is an instruction from for example an imaging menu or a user. Therefore, in the present exemplary embodiment, the correspondence relationships described above are obtained in advance for each of the detection sensitivities (modes). Note that setting of the detection sensitivities (modes) may be performed by pre-providing a setting section (not illustrated in the drawings) in the electronic cassette 20, and then performing setting in such a setting section. Configuration may be made such that the mode is decided by determining whether or not there is an instruction from for example an imaging menu or a user.

Correspondence relationships between the imaging subject data and the detection limit include, for example, correspondence relationships between the body thickness of the investigation subject and a detection limit radiation amount (for example the lowest limit value of the radiation amount of the radiation X that needs to be irradiated from the radiation generation device 12 in order to cause a detectable radiation amount of the radiation X to arrive at the electronic cassette 20). In such cases, when the body thickness of the imaging subject is contained in the imaging subject data, the detection limit radiation amount corresponding to such a body thickness is derived based on the stored correspondence relationships (correspondence relationships according to detection sensitivities). Moreover, in cases in which the height and weight of the investigation subject 30 is contained therein, the body thickness is computed from the height and the weight. There are no particular limitations to the manner in which the body thickness is computed, and any existing method may be employed. Note that the body thickness of the imaging subject is influenced by such factors as the site of the imaging subject and the age and gender of the investigation subject 30, and hence a more appropriate computation of the body thickness of the imaging subject is enabled by computation with these factors added to the imaging subject data.

Moreover, examples of the correspondence relationships between irradiation data and detection limit include for example correspondence relationships between radiation amount of radiation X irradiated from the radiation generation device 12 and the body thickness of the investigation subject that is the detection limit (the upper limit value of the body thickness of the investigation subject). In such cases, when the radiation amount is contained in the irradiation data, the body thickness that is the detection limit corresponding to the radiation amount is derived based on the stored correspondence relationships (correspondence relationships according to detection sensitivities).

Moreover, examples of correspondence relationships between imaging subject data and irradiation data and detection limits include for example correspondence relationships (correspondence relationships according to detection sensitivities) between the body thickness of the imaging subject, the separation distance between the radiation irradiation source 22A and the investigation subject 30, and the radiation amount that is the detection limit (for example the lower limit value of the radiation amount of the radiation X that needs to be irradiated from the radiation generation device 12 in order to cause a detectable radiation amount of the radiation X to arrive at the electronic cassette 20).

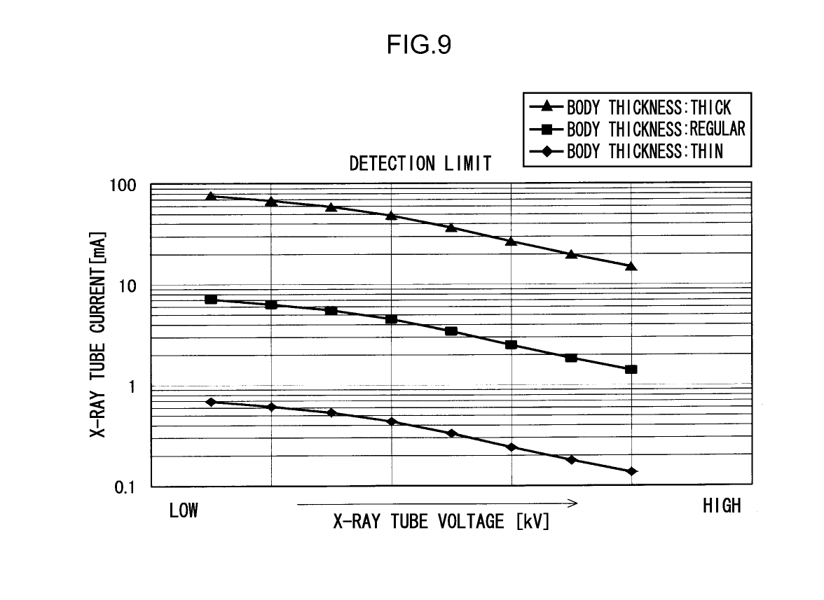

Moreover, for example, an example follows of correspondence relationships between imaging subject body thickness and the X-ray tube current and X-ray tube voltage of the radiation irradiation source 22A at the detection limit (correspondence relationships according to detection sensitivities). FIG. 9 illustrates a graph showing specific examples of correspondence relationships in the normal sensitivity mode between imaging subject body thickness and X-ray tube current and X-ray tube voltage of the radiation irradiation source 22A at the detection limit. Note that in FIG. 9, the body thickness “regular” refers to a case of a regular (average) body thickness for an ordinary investigation subject 30. Moreover the body thickness “thick” refers to a case in which the body thickness is greater than regular. The body thickness “thin” refers to a case in which the body thickness is thinner than regular. Note that although specific illustration, such as that in FIG. 9, is omitted for correspondence relationships in the high sensitivity mode between imaging subject body thickness and the X-ray tube current and X-ray tube voltage of the radiation irradiation source 22A at the detection limit, the radiation amount (X-ray tube current and X-ray tube voltage) at the detection limit corresponding to a given body thickness is smaller (less) in the high sensitivity mode. Moreover, the body thickness at the detection limit corresponding to a given radiation amount is thicker in the high sensitivity mode.

Note that the detection limit calculation method is not limited thereto and furthermore, for example, a table may be provided, and calculation may be made from the graph or calculated from the table. Moreover, a relationship equation expressing the corresponding relationships referred to above may be obtained in advance, such as by experimentation, and then employed for calculation.

Note that the detection limit to be calculated is not limited to the radiation amount and the body thickness as described above, and may be detection sensitivity required for capturing an appropriate radiographic image. In the present exemplary embodiment explained above, the corresponding relationships are obtained for each of the detection sensitivities, and so the detection sensitivity that is required according to imaging subject data and irradiation data may be calculated similarly to in the above cases.

At the next step S110 determination is made as to whether or not to notify the user of the calculated detection limit. Determination of whether or not to notify the detection limit may be performed by receiving in advance a setting of whether or not a user is to be notified from for example the radiographic image processing apparatus 14 or the radiographic image reading apparatus 18, through the I/F section 68, and storing this setting for example in the radiographic image processing apparatus 14. Moreover, in cases in which such a setting is included in an image capture menu, the setting may be acquired during image capture menu reception, or may be pre-set so as to automatically notify. Processing proceeds to step S116 in cases in which no notification of the detection limit is made.

However, affirmative determination is made in cases in which notification of the detection limit is made, and processing proceeds to step S112 where the detection limit is notified. In the present exemplary embodiment, notification is made through the I/F section 68 to the notification destination, such as the display 50 of the console 16 or the display 23 of the radiographic image reading apparatus 18, preset similarly to the setting of whether or not to make notification. Thus when the detection limit has been notified, a user is able to determine based on the notified detection limit whether or not the imaging subject conditions (for example the body thickness) and the irradiation conditions (for example, the radiation amount, the X-ray tube voltage or the X-ray tube current) exceed the detection limit, enabling determination as to whether or not detection will be possible. For example, there is a high possibility of detection not being possible in cases in which a planned radiation amount (or the X-ray tube voltage or the X-ray tube current) for irradiating from the radiation generation device 12 onto the imaging subject is less than the radiation amount (or the X-ray tube voltage or the X-ray tube current) notified as the detection limit. Moreover, for example, determination may be made as to whether or not the body thickness of the imaging subject exceeds the body thickness notified as the detection limit, enabling determination to be made as to whether or not detection is possible. For example, there is a high possibility of detection not being possible in cases in which the body thickness of the imaging subject is greater than the body thickness notified as the detection limit. In either of these cases, there is a high possibility of detection not being possible due to the radiation amount reaching the electronic cassette 20 being small. Accordingly, in order to make detection possible, measures may be taken such as for example increasing the radiation amount that reaches the electronic cassette 20, or employing a high sensitivity for the sensitivity (detection sensitivity) of the electronic cassette 20. In the present exemplary embodiment, configuration is made such that the radiation amount and the sensitivity of the electronic cassette 20 can be changed on instruction from the user.

In cases in which the radiation amount is changed, the radiation generation device 12 is controlled through the radiation controller 62 to change the radiation amount that reaches the electronic cassette 20 based on the change instruction received from for example the console 16 or the radiographic image reading apparatus 18. Note that in cases in which the radiation amount of the radiation X irradiated from the radiation generation device 12 is increased, since the radiation exposure of the investigation subject 30 is increased and there is an influence on the captured radiographic images, configuration is preferably made such that for example an upper limit value to the radiation amount is set in advance and change is only possible within a range that does not exceed the upper limit value.

In cases in which the detection sensitivity of the electronic cassette 20 is changed, the electronic cassette 20 is controlled through the panel controller 64 to change the detection sensitivity based on the change instruction received for example from the console 16 or the radiographic image reading apparatus 18. An example of a method to change the detection sensitivity of the electronic cassette 20 includes changing the bias voltage applied to the sensor portions 103. In such cases, the larger the bias voltage the higher the detection sensitivity since reading charge from the pixels 100 becomes easier. Moreover, for example the gain (amplification ratio) of the amplification circuits 120 may be changed. In such cases, the larger the gain the higher the detection sensitivity, since the electrical signal becomes larger. A further example follows of changing the sampling frequency of charges (electrical signals) accumulated in the pixels 100. In such cases, the detection sensitivity is raised the lower the sampling frequency. Note that changing the detection sensitivity may be accomplished by instructing so as to switch between the normal sensitivity mode and the high sensitivity mode of the electronic cassette 20. Note that in cases in which the electronic cassette 20 has even more plural modes (detection sensitivities), such as a low sensitivity mode, an instruction may be given so as to switch to a higher sensitivity mode than the current detection sensitivity. Note that out of changing radiation amount and changing detection sensitivity, preferably change to the detection sensitivity is prioritized from the perspective of suppressing the exposure amount to the investigation subject 30. Thus in the present exemplary embodiment, configuration is made such that the detection sensitivity of the electronic cassette 20 is changed from the normal sensitivity mode to the high sensitivity mode in cases such as those in which the thickness of the imaging subject exceeds the detection limit, cases in which the tube voltage of the radiation irradiation source 22A is lower than the detection limit, and cases in which the tube current is lower than the detection limit.

At step 114, determination is made as to whether or not a user has made an instruction such as one of those described above. Negative determination is made in the absence of such an instruction, and processing proceeds to step S116. However, affirmative determination is made in cases in which there is an instruction, processing returns to step S108, and the detection limit corresponding to the instructed conditions is recalculated, and the present processing repeated.

At the next step S116, determination is made as to whether or not irradiation start detection is possible. For example, in cases in which the detection limit is a radiation amount then determination as to whether or not detection is possible is made based on the acquired irradiation data. Moreover, for example, in cases in which the detection limit is a body thickness, then body thickness is derived based on the acquired imaging subject data, and then determination is made as to whether or not detection is possible. Moreover, for example, in cases in which there is a detection limit at a detection sensitivity (mode), the detection sensitivity (mode) at the detection limit is compared with the currently set detection sensitivity (mode), and determination is made as to whether or not detection is possible, by determining whether or not the currently set detection sensitivity (mode) has a higher sensitivity. Affirmative determination is made and processing proceeds to step S118 in cases in which detection is possible, radiographic image capture processing is performed, then the present processing is ended after a radiographic image of the imaging subject has been captured.

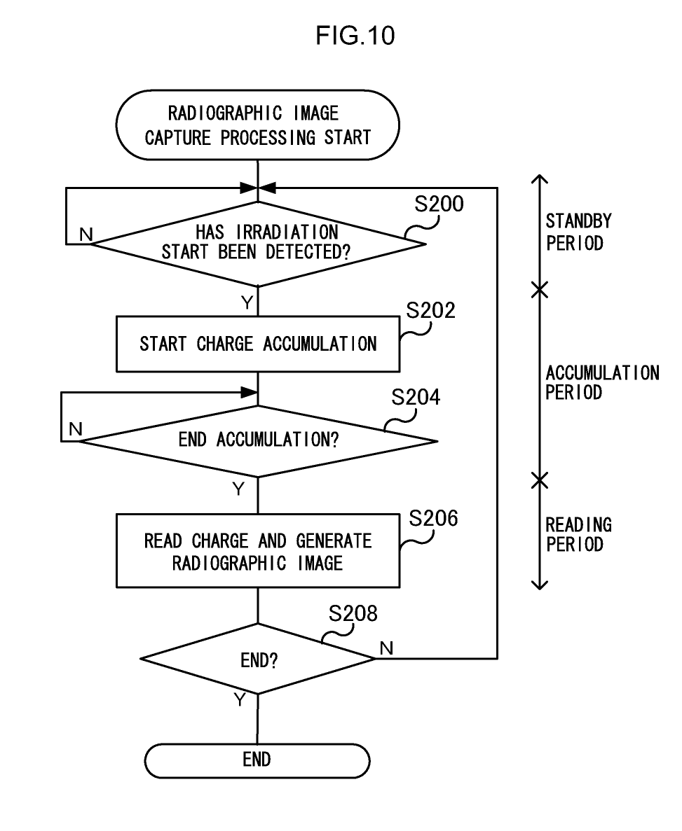

Explanation follows regarding the radiographic image capture processing of the present exemplary embodiment. FIG. 10 is a flow chart illustrating an example of flow in radiographic image capture processing in the electronic cassette 20 of the present exemplary embodiment. The electronic cassette 20 of the present exemplary embodiment captures a radiographic image by detecting radiation X irradiation start, accumulating charges in each of the pixels 100 of the radiation detection device 26, and then generating a radiographic image based on image data corresponding to the accumulated charges.

At the start of image capture, the electronic cassette 20 transitions to a standby period in which radiation X irradiation start detection is performed. At step S200 determination is made as to whether or not radiation X irradiation start has been detected.

When radiation is irradiated from the radiation generation device 12, the irradiated radiation X is absorbed by the scintillator and converted into visible light. The light converted into visible light by the scintillator is irradiated onto the sensor portions 103 of each of the pixels 100. Charges are generated in the sensor portions 103 when the light is irradiated. The generated charges are collected in the lower electrodes 81.

In the radiographic image capture pixels 100A, the charges collected in the lower electrodes 81 are accumulated since the drain electrode 83 and the source electrode 79 are not shorted. However, in the radiation detection pixels 100B, the charges collected in the lower electrodes 81 flow out into the signal lines 73 since the drain electrode 83 and the source electrode 79 are shorted.

In the electronic cassette 20 of the present exemplary embodiment, as described above, the electrical signals (charge data) output from the radiation detection pixels 100B are detected in the amplification circuits 120 of the signal detection circuit 105, the controller 106 compares the detected electrical signals (charge data) against predetermined specific values for detection, and detection of radiation X irradiation start is made by whether or not the specific value or greater has been reached. Negative determination is made when radiation X irradiation start is not detected, and a standby state is adopted. However, affirmative determination is made when irradiation start is detected, and processing proceeds to step S202, and the electronic cassette 20 transitions to a charge accumulation period for accumulating charges. Accordingly, at step S202, accumulation of charges generated according to the irradiated radiation X is started in each of the pixels 100.

The radiographic image capture pixels 100A of the radiation detection device 26 are in a charge accumulated state since the TFT switches 74 are still in the OFF state. However, the radiation detection pixels 100B output charges to the signal detection circuit 105 even during the charge accumulation period (the OFF state of the TFT switches 74) since the TFT switches 74 are shorted. The S/H switch SW is switched ON/OFF at specific timings, and data of the charges output from the radiation detection pixels 100B is input as electrical signals (charge data) to the controller 106 through the amplification circuits 120 and the ADC 124 of the signal detection circuit 105.

At the next step S204, determination is made as to whether or not to end charge accumulation. There is no particular limitation to the method to determine whether or not to end charge accumulation, and for example determination may be made depending on whether or not a specific duration has elapsed since accumulation start. Negative determination is made when not complete, and charge accumulation is continued. However, affirmative determination is made when complete, and processing proceeds to step S206. At step S206 transition is made to a reading period, charges are read from the pixels 100, a radiographic image is generated and output based on the read charges. Note that during the reading period, specifically the TFT switches 74 of the pixels 100A are switched ON in sequence by applying an ON signal through the gate lines 101 in sequence to the gate electrodes 72 of the TFT switches 74. The charges are read by outputting electrical signals corresponding to the charge amount accumulated in each of the pixels 100A to the signal lines 73.

At the next step S208 determination is made as to whether or not to end image capture. Negative determination is made in cases in which successive image capture is performed, such as in video image capture, and processing returns to step S200 where the present processing is repeated. However, affirmative determination is made when image capture is to be ended and the present processing is ended. Thus the electronic cassette 20 of the present exemplary embodiment performs image capture of 1 frame (one image) of radiographic image using the standby period waiting to detect radiation X irradiation start, the accumulation period in which each of the pixels 100A accumulates charges generated according to the irradiated radiation X, and the reading period in which the accumulated charges are read.

However, negative determination is made in cases in which determination at step S116 is that detection is not possible, and processing proceeds to step S120. At step S120 notification is made to a user that detection is not possible. Note that notification that detection is not possible may be performed similarly to notification of the detection limit (see step S112). At the next step S122, determination is made as to whether or not to change the detection sensitivity or the irradiation conditions. Note that determination as to whether or not to make such changes may be performed similarly to the determination of step S114. In cases in which no change is to be made, the present processing is ended without capturing a radiographic image since there is concern that under the current conditions appropriate detection of radiation X irradiation start will not be made by the electronic cassette 20, leading to unnecessary radiation exposure of the investigation subject 30. However, affirmative determination is made in cases in which a user instructs a change on receiving the notification of step S120, and in cases preset so as to perform a change, and processing proceeds to step S124. At step S124, after instructing change of the detection sensitivity and/or change of the irradiation conditions, processing returns to step S108, and the present processing is repeated.

As explained above, in the radiographic image capture system 10 of the present exemplary embodiment, during radiographic image capture, the radiation X irradiation start is detected by the electronic cassette 20 itself, and accumulation of the charges generated in the sensor portions 103 is started. Moreover, the radiographic image processing apparatus 14 calculates a detection limit for irradiation start in the electronic cassette 20 based on the imaging subject data or the irradiation data or both, and performs notification thereof. Moreover, determination is made as to whether or not detection is possible of radiation X irradiation start in the electronic cassette 20 based on the calculated detection limit, and the determination result notified. In cases in which it is determined that detection is not possible, the detection sensitivity is changed to give a higher sensitivity or the radiation amount of the radiation X to be irradiated by the radiation generation device 12 is increased, according to settings made by the user or a pre-instructed setting.

Consequently, it is possible to suppress cases of detection not being possible due to the radiation amount of the radiation X reaching the electronic cassette 20 even though the radiation X is being irradiated onto the investigation subject 30, cases in which radiographic image capture is not performed. The duration for the detection of radiation X irradiation start, which would increase the radiation exposure amount of the investigation subject 30, can also be suppressed. Consequently, unnecessary radiation exposure of the investigation subject 30 can be suppressed.

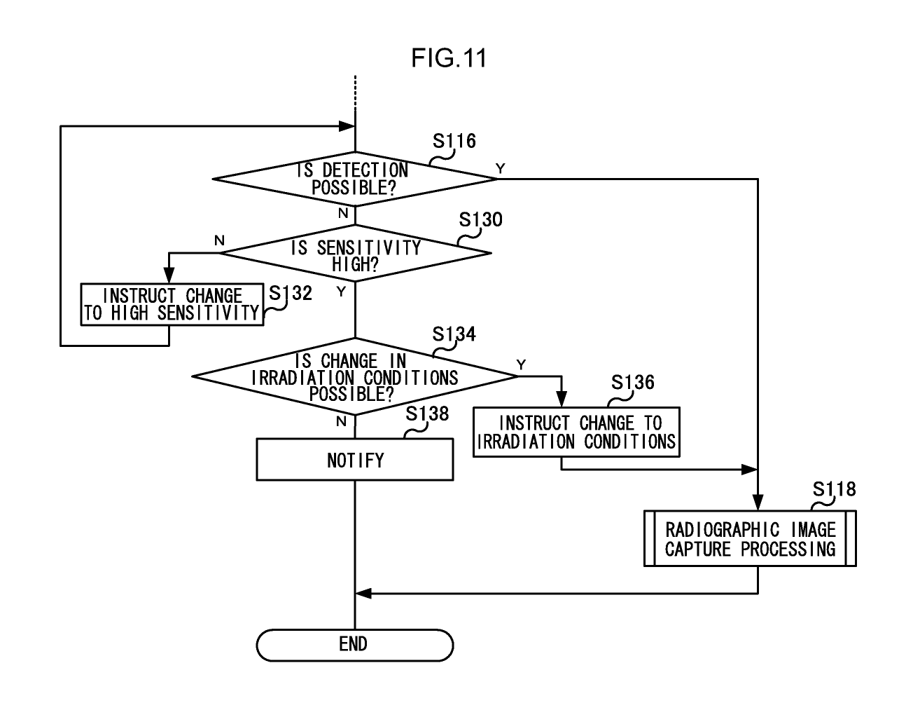

Note that as described above, in cases in which radiation start detection is not possible (step S116=N), after notifying this fact (step S120), the detection sensitivity and/or the irradiation conditions are changed (step S122 and step S124), based on instruction from the user, however there is no limitation thereto, and configuration may be made such that the detection sensitivity and/or the irradiation conditions are changed automatically. A flow chart of an example of flow of detection limit calculation processing in the radiographic image capture system 10 in such cases is illustrated in FIG. 11. Note that detailed explanation is omitted of processing similar to that described above for the detection limit calculation processing (see FIG. 8) and the radiographic image capture processing (see FIG. 10). As illustrated in FIG. 11, at step S116 of the detection limit calculation processing, in cases in which it has been determined that detection is not possible, processing proceeds to step S130. At step S130, determination is made as to whether or not as to whether or not a high sensitivity mode is set for the detection sensitivity (mode) of the electronic cassette 20. Negative determination is made in cases in which a high sensitivity mode is not set, namely, in cases in which a normal sensitivity mode is set in the present embodiment, and processing proceeds to step S132, then after instruction to make the sensitivity of the electronic cassette 20 a high sensitivity, processing returns to step S116, and determination is performed again as to whether or not irradiation start detection is possible. Note that similarly to as described above, the method of changing the detection sensitivity may for example be to change the bias voltage, the gain (amplification ratio) of the amplification circuits 120 or sampling frequencies. Namely, affirmative determination is made when a high sensitivity is set as the detection sensitivity of the electronic cassette 20, processing proceeds to step S134, and then determination is made as to whether or not as to whether it is possible to change the irradiation conditions. For example, in cases in which the radiation amount of the radiation X irradiated by the radiation generation device 12 is changed according to the calculated detection limit so as to make irradiation start detection possible, since there is generally a predetermined limit value (radiation amount range) of radiation amounts of radiation X that can be irradiated, the radiation amount cannot be changed to exceed this limit value. Affirmative determination at step 134 is made in cases in which the desired change in radiation amount is within the range of irradiation possible radiation amounts, since it is possible to change the irradiation conditions, and processing proceeds to step S136. At step S136, after the irradiation condition change instruction has been output to the radiation generation device 12, processing proceeds to step S118, then the present processing is ended after radiographic image capture processing has been performed as described above. As a specific example, instruction is given to increase the radiation amount of radiation X irradiated by the radiation generation device 12 so as to make detection of irradiation start possible. Accordingly, since the radiation amount of the radiation X reaching the electronic cassette 20 reaches a radiation amount capable of detection, appropriate radiographic image capture processing can be performed at step S118. However, for example in cases in which the desired change in radiation amount reaches the limit value, determination is made at step S134 that it is not possible to change the irradiation conditions, negative determination is made and processing proceeds to step S138. At step S138, the fact that radiation X irradiation start cannot be detected even if the detection sensitivity or the irradiation conditions are changed is notified to the user, and then the present processing is ended. Due to having such a configuration, the detection sensitivity and the irradiation conditions can be automatically changed such that detection of radiation X irradiation start is made possible. Note that in the present exemplary embodiment, although explanation has been given of a case in which a change in detection sensitivity is prioritized above a change in irradiation conditions, there is no limitation thereto. Although it is preferable to prioritize a change to the detection sensitivity from the perspective of suppressing the radiation exposure of the investigation subject 30, change to the irradiation conditions may be prioritized.

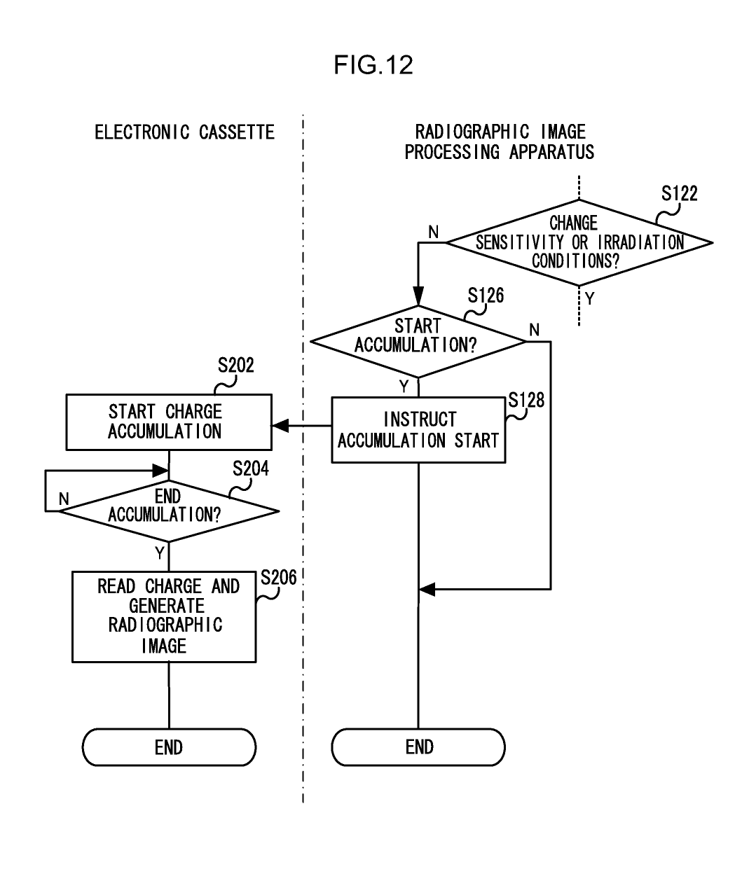

Moreover, in the example described above, in a case in which detection of the radiation X irradiation start (step S116=N) is not possible, then after notifying this fact (step S120), in cases in which there is no change to either the detection sensitivity or the irradiation conditions (step S122=N) the present processing is ended. However, in cases in which emphasis is placed on radiographic image capture, configuration may be made to not end the present processing and to forcibly capture a radiographic image. FIG. 12 illustrates a flow chart of an example of flow of processing in radiographic image capture with the radiographic image capture system 10 in such a case. Note that detailed explanation is omitted of processing similar to that described above for the detection limit calculation processing (see FIG. 8) and the radiographic image capture processing (see FIG. 10). As illustrated in FIG. 12, in step S122 of the detection limit calculation processing, in cases in which neither the detection sensitivity nor the irradiation conditions are changed processing proceeds to step S126, and determination is made as to whether or not to start charge accumulation in the electronic cassette 20. In cases in which image capture is forcibly started irrespective of whether or not radiation X irradiation start has been detected in the electronic cassette 20, the radiographic image processing apparatus 14 instructs the electronic cassette 20 to transition to the accumulation period of the radiographic image capture processing described above. Then affirmative determination is made in cases in which instruction is made from for example the console 16 or the radiographic image reading apparatus 18 through the I/F section 68 so as to execute image capture, and processing proceeds to step S128, then after charge accumulation start has been instructed to the electronic cassette 20, the present processing is ended. Moreover, negative determination is made at step S126 in cases in which instruction is not given to execute image capture, and the present processing is ended. However, in the electronic cassette 20 instructed to start charge accumulation, step S202 to step S206 of the radiographic image capture processing described above is executed and radiographic image capture is performed. Specifically, transition is made to the accumulation period when an instruction for accumulation start has been received, accumulation of charges generated in the sensor portions 103 according to the irradiated radiation X is started (step S202), and accumulation continues until accumulation is ended (step S204=N). When accumulation is ended (step S204=Y), transmission is made to the reading period, and the TFT switches 74 are driven to read the charges. Then after a radiographic image has been generated and output according to the charges read (step S206), the present processing is ended. Note that configuration may be made such that in cases in which the detection sensitivity or the irradiation conditions are changed automatically (see FIG. 11), the fact that detection of irradiation start cannot be made even when the detection sensitivity and the irradiation conditions are changed is notified to the user (step S138), and then radiographic image capture is forcibly performed in a similar manner.

Moreover, in the present exemplary embodiment, although explanation has been given of a case in which the radiographic image processing apparatus 14 functions as a detection limit calculation device that calculates the detection limit of the electronic cassette 20, there is no limitation thereto. For example, configuration may be made such that the electronic cassette 20 calculates the detection limit itself, or configuration may be made such that for example the console 16 functions as a detection limit calculation device.

Moreover, in the present exemplary embodiment, although explanation has been given of a case in which a detection limit arising from the radiation amount of the radiation X irradiated (arriving) at the electronic cassette 20 being too small, there is no limitation thereto. For example, configuration may be made so as to calculate a detection limit arising from the radiation amount of the radiation X irradiated (arriving) at the electronic cassette 20 being too great.

Moreover, in the present exemplary embodiment, explanation has been given of a case in which a detection limit is calculated in cases in which the radiation X irradiation start is detected, however configuration may similarly be made such that the electronic cassette 20 itself calculates a detection limit for irradiation stop in cases in which radiation X irradiation stop is detected.

Moreover, in the present exemplary embodiment, explanation has been given of cases in which the detection limit is calculated based on the imaging subject data or the irradiation data or both, however there is no limitation thereto, and configuration may be made such that the detection limit is calculated based on further other data. For example, configuration may be made such that the detection limit is calculated according to the type of the radiographic image, such as whether or not the radiographic image for capture is a video image or a still image.

Moreover, the configuration and method for detecting the radiation X irradiation start using the electronic cassette 20 itself are not limited to those of the present exemplary embodiment. For example, although explanation has been given above wherein pixels equipped with TFT switches 74 with shorted sources and drains are employed as the radiation detection pixels 100B, there is no limitation thereto. For example, a connection line may be formed from partway along the drain electrode 83, so as to connect to the signal lines 73. In such cases too, the sources and drains of the TFT switches 74 are effectively shorted. Moreover, in cases in which the sources and drains of the TFT switches 74 are shorted, the gate electrode 72 may be formed separated from the respective gate lines 101. Moreover, for example, configuration may be made such that there is an electrical disconnect between the drain electrode 83 and the contact hole 87 in the radiation detection pixels 100B by connecting together the sensor portions 103 and the signal lines 73 through a connection line 82 and the contact hole 87.

Moreover, in the present exemplary embodiment, although explanation has been given of a case in which pixels with shorted TFT switches 74 are employed as the radiation detection pixels 100B, there is no particular limitation to the radiation detection pixels 100B. For example, pixels that do not have shorted TFT switches 74 may be employed as the radiation detection pixels 100B. In such cases, control of the TFT switches 74 of the radiation detection pixels 100B is performed independently of control of the TFT switches 74 of the pixels 100A. Moreover, the pixels 100B in such cases may be employed as specific pixels 100 of the radiation detection device 26, or may be provided as different pixels to the pixels 100 in the radiation detection device 26.

Moreover, in the radiation detection device 26 of the electronic cassette 20 of the present exemplary embodiment (see FIG. 3), the radiation detection pixels 100B are connected to some of the signal lines 73, however there is no limitation thereto. Configuration may be made such that there are radiation detection pixels 100B at positions connected to all of the signal lines 73, and there is no particular limitation to the positions where the radiation detection pixels 100B are provided.

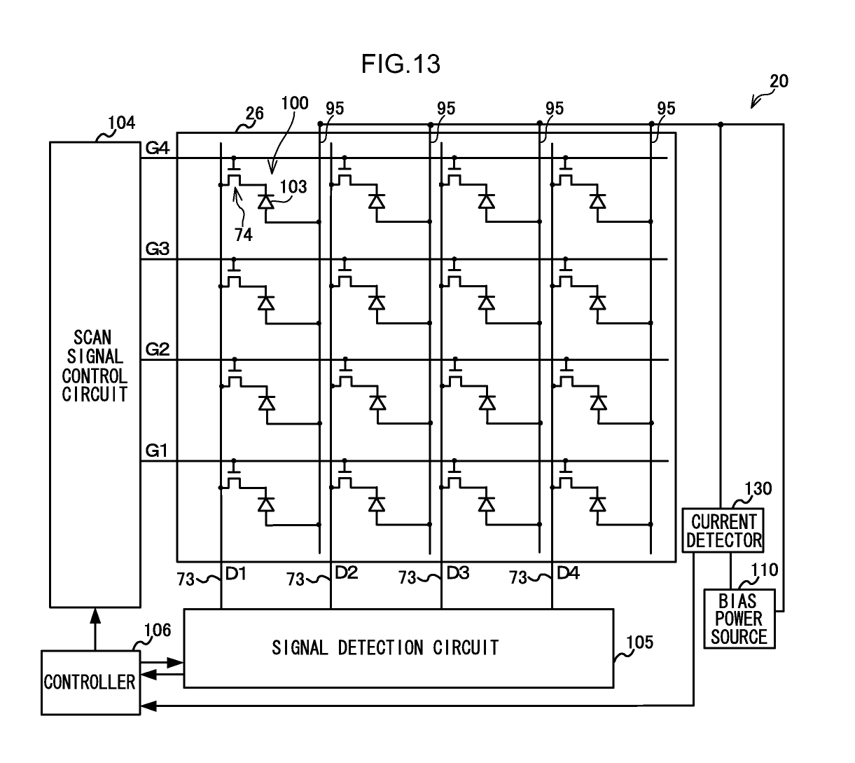

Moreover, an explanation has been given in the present exemplary embodiment of a method for detecting radiation X irradiation start using the electronic cassette 20 itself, in a case in which radiation irradiation start is detected based on the charge generated by the radiation detection pixels 100B, however there is no limitation thereto. For example, the radiation irradiation start may be detected by the electronic cassette 20 based on for example charges flowing in the common electrode lines 95. FIG. 13 is a configuration diagram illustrating an example of an overall configuration of an electronic cassette 20 in a case in which radiation irradiation start is detected based on for example charges flowing in the common electrode lines 95. As illustrated in FIG. 13, in such cases, the electronic cassette 20 is not equipped with radiation detection pixels 100B and all the pixels are of a similar configuration. Moreover, in the electronic cassette 20, common electrode lines 95 are connected to a bias power source 110 through a current detector 130. In the electronic cassette 20 illustrated in FIG. 13, in order to apply a bias voltage to each of the pixels 100, the bias voltage is applied to each of the pixels 100 directly, and not through the current detector 130.

The current detector 130 includes a function to detect current flowing in from each of the pixels 100 through the common electrode lines 95. A controller 106 compares a current value of current flowing in the common electrode lines 95 detected by the current detector 130 with a predetermined threshold value employed for detection, and detects radiation irradiation start by whether or not it is the threshold value or greater. When radiation is being irradiated onto a radiation detection device 26 and charges are generated in sensor portions 103 of the pixels 100, current flows in each of the common electrode lines 95 according to the generated charge (charge amount). Thus in the present exemplary embodiment a relationship is obtained in advance between the current value of current flowing in the common electrode lines 95 and the radiation amount irradiated onto the radiation detection device 26, and a current value to use to detect irradiation start is predetermined as a threshold value. Note that since the current value of current flowing in the common electrode lines 95 increases as the charges generated by the sensor portions 103 (charge amount) increases, the current value of current flowing in the common electrode lines 95 also increases with an increase in the radiation amount of radiation X irradiated. A detection threshold value (current value) is obtained in advance such as by experimentation, and then the controller 106 detects radiation X irradiation start in cases in which the current value of the current flowing in the common electrode lines 95 detected by a pseudo-current detector 122 is the threshold value or greater. Note that in order to detect such current flowing in the common electrode lines 95, current flowing in the common electrode lines 95 may be detected in a state in which respective TFT switches 74 of the respective pixels 100 are switched OFF. Moreover, the TFT switches 74 may be temporarily placed in an ON state, so as to detect the current flowing in the common electrode lines 95.

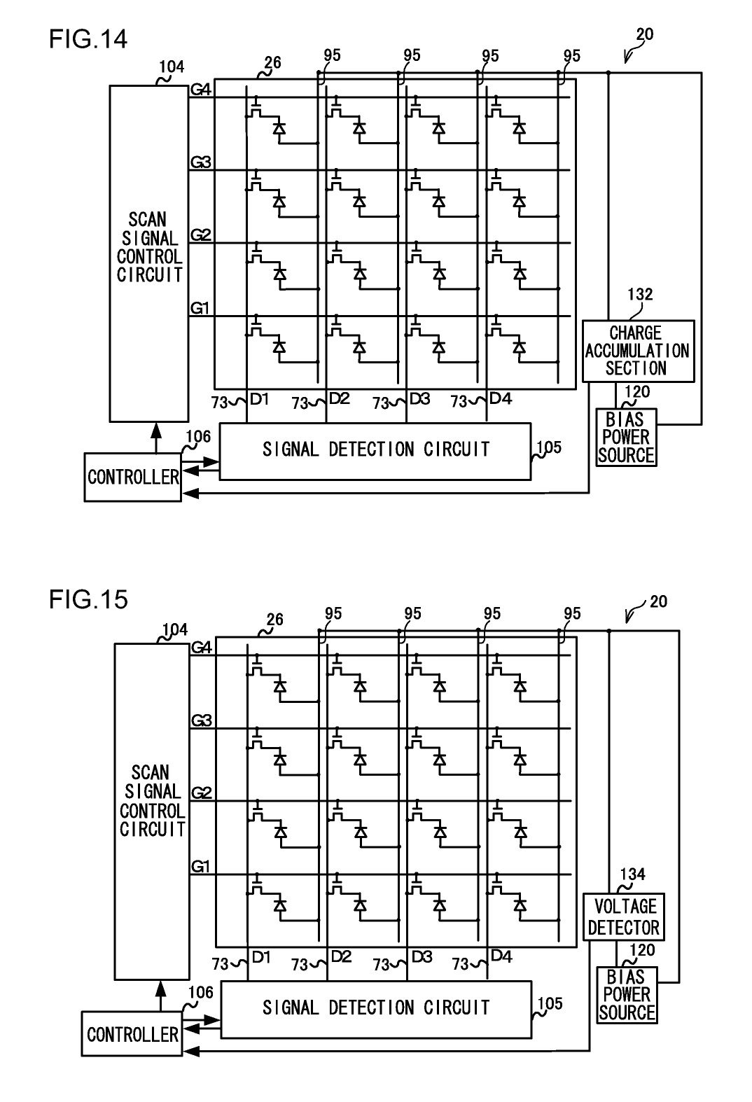

Note that although explanation has been given of a case in which the current value of current flowing in the common electrode lines 95 is detected by the current detector 130, there is no limitation thereto. For example, as illustrated in FIG. 14, configuration may be made such that charges flowing in the common electrode lines 95 are accumulated in a charge accumulation section 132, and the radiation X irradiation start is detected based on the accumulated charge amount. Moreover, configuration may be made such that, as illustrated in FIG. 15, the voltage of current flowing in the common electrode lines 95 is detected by a voltage detector 134, and radiation irradiation start is detected based on the detected voltage value. Moreover, although explanation has been given above of a case in which radiation X irradiation start is detected based on current flowing in all of the common electrode lines 95, there is no limitation thereto, and configuration may be made such that radiation X irradiation start is detected based on current flowing in only some of the common electrode lines 95.

As another method for detecting radiation X irradiation start with the electronic cassette 20 itself, configuration may be made such that, for example, a current detector 130 is provided in the scan signal control circuit 104, and the radiation X irradiation start is detected based on changes in the current flowing in gate lines 101. Configuration may also be made such that, for example, a current detector 130 or the like is provided in the signal detection circuit 105, and the radiation X irradiation start is detected based on changes in the current flowing in the signal lines 73. Moreover, configuration may be made such that for example separate sensors are provided for radiation detection, and the radiation irradiation start is detected by the electronic cassette 20 itself based on the detection results of the sensors.

Moreover, in the present exemplary embodiment, the sensitivity to capture an appropriate radiographic image is taken as the detection sensitivity for detecting radiation X irradiation start, however there is no limitation thereto. Configuration may be made such that the sensitivity for capturing radiographic images and the detection sensitivity for detecting radiation X irradiation start are set separately to each other. In such cases, a detection limit may be calculated based on the detection sensitivity to detect radiation X irradiation start. Moreover, the detection sensitivity when detecting radiation X irradiation start may be made different from the sensitivity when performing radiographic image capture. For example, configuration may be made such that after detecting the radiation X irradiation start in high sensitivity mode, a switch is made to the normal sensitivity mode, and then radiographic image capture (accumulation of charges for image capture) is performed.

Moreover, in the present exemplary embodiment, explanation has been given of a case in which the present invention is applied to the indirect conversion method radiation detection device 26 that converts converted light into charge, however there is no limitation thereto. For example, the present invention may be applied to a direct conversion method radiation detection device that employs a material to convert the radiation X directly into charge, such as amorphous selenium as a photoelectric conversion layer to absorb radiation and convert the radiation into charge.

Moreover, in the present exemplary embodiment, explanation has been given of a case in which the present invention is applied to a radiographic imaging apparatus (radiographic image capture system 10) that captures a radiographic image of an imaging target site of an investigation subject 30 on an imaging table 32 as an imaging subject, however the radiographic imaging apparatus is not particularly limited. For example, a radiographic image may be captured with the breast of the investigation subject 30 as the imaging subject, in application to what is referred to as mammography. Moreover, although explanation has been given in the present exemplary embodiment to a human as the investigation subject 30, application may be made, for example, to another animal.

Moreover, the configuration and operation of the radiographic image capture system 10, the radiation generation device 12, the radiographic image processing apparatus 14, the console 16, the electronic cassette 20 and the radiation detection device 26 etc. explained in the present exemplary embodiment are merely examples thereof, and obviously modifications are possible according to the circumstances within a range not departing from the scope of the present invention.

Moreover, there are no particular limitations to the radiation X in the present exemplary embodiment, and application can be made of for example X-rays and gamma rays.

The calculation unit of the detection limit calculation device of the present invention may be configured wherein the calculation unit calculates whether or not detection by the detection unit is possible based on both the imaging subject data and the irradiation data.

The imaging subject data of the present invention preferably includes at least one factor selected from the group consisting of a thickness of the imaging subject, a height and weight of the imaging subject, an image capture site of the imaging subject, a size of the image capture site of the imaging subject, and a shape of the image capture site of the imaging subject.

The detection limit calculation device of the present invention preferably includes a change unit that changes a detection sensitivity of the detection unit based on a calculation result of the calculation unit.

The detection limit calculation device of the present invention may be configured such that: the calculation result of the calculation unit is a detection sensitivity limit that is a detection limit; the detection limit calculation device includes a comparison unit that compares the detection sensitivity limit and the current detection sensitivity of the detection unit; and the change unit changes the detection sensitivity of the detection unit based on the comparison result of the comparison unit.

The detection limit calculation device of the present invention preferably includes a calculation result notification unit that notifies a calculation result of the calculation unit.

The detection limit calculation device of the present invention may include an imaging subject data reception unit that receives the imaging subject data.

The detection limit calculation device of the present invention may include an irradiation data reception unit that receives the irradiation data.

The detection unit of the detection limit calculation device of the present invention may be configured wherein the detection unit detects as radiation irradiation start in a case in which a change with time of a radiation amount of irradiated radiation satisfies a specific irradiation detection condition.

The specific irradiation detection condition of the present invention may include a case in which a change amount of a radiation amount per unit time has exceeded a threshold value, or a case in which a number of times a change amount of a radiation amount per unit time is a threshold value or greater is a predetermined number of times or greater, or both cases.

The detection limit calculation device of the present invention may further include a control unit that controls the image capture unit, that accumulates charge according to the radiation irradiated and that generates a radiographic image based on accumulated charges, by controlling such that charge accumulation is performed irrespective of a detection result of the detection unit.

The control unit of the detection limit calculation device of the present invention may further include a notification unit that notifies that the control unit is controlling such that charge accumulation is performed in the image capture unit.

The image capture unit of the present invention may be configured to include a radiation detection device that contains plural pixels, each including respective sensor portions that generate charges according to a radiation amount of irradiated radiation and respective switching elements that read charges from the sensor portions and output to signal lines electrical signals that accord with the charges, and common electrode lines that supply a bias voltage to the sensor portions; and the detection unit of the detection limit calculation device of the present invention detects that irradiation of radiation has started in cases in which an electrical signal that arises from charges generated in the sensor portion and that flows in the common electrode line satisfies a specific irradiation detection condition.

According to the present invention, the advantageous effect can be obtained of enabling unnecessary radiation exposure to the imaging subject to be suppressed.

What is claimed is:

1. A detection limit calculation device comprising:
a calculation unit that calculates, for image capture of a radiographic image of an imaging subject using an image capture unit, a detection limit of a detection unit that detects whether irradiation of radiation has started based on a radiation amount of radiation comprising radiation that has been irradiated and passed through the imaging subject, based on imaging subject data relating to the imaging subject or irradiation data relating to irradiation of radiation or both thereof.

2. The detection limit calculation device of claim 1, wherein the calculation unit calculates whether or not detection by the detection unit is possible based on both the imaging subject data and the irradiation data.

3. The detection limit calculation device of claim 1, wherein the imaging subject data includes at least one factor selected from the group consisting of a thickness of the imaging subject, a height and weight of the imaging subject, an image capture site of the imaging subject, a size of the image capture site of the imaging subject, and a shape of the image capture site of the imaging subject.

4. The detection limit calculation device of claim 1, further comprising a change unit that changes a detection sensitivity of the detection unit based on a calculation result of the calculation unit.

5. The detection limit calculation device of claim 4, wherein:
a calculation result of the calculation unit is a detection sensitivity limit that is a detection limit;
a comparison unit is provided to compare the detection sensitivity limit and the current detection sensitivity of the detection unit; and
the change unit changes the detection sensitivity of the detection unit based on the comparison result of the comparison unit.

6. The detection limit calculation device of claim 1, further comprising a calculation result notification unit that notifies a calculation result of the calculation unit.

7. The detection limit calculation device of claim 1, further comprising an imaging subject data reception unit that receives the imaging subject data.

8. The detection limit calculation device of claim 1, further comprising an irradiation data reception unit that receives the irradiation data.

9. The detection limit calculation device of claim 1, wherein the detection unit detects as radiation irradiation start in a case in which a change with time of a radiation amount of irradiated radiation satisfies a specific irradiation detection condition.

10. The detection limit calculation device of claim 9, wherein the specific irradiation detection condition includes a case in which a change amount of a radiation amount per unit time has exceeded a threshold value, or a case in which a number of times a change amount of a radiation amount per unit time is a threshold value or greater is a predetermined number of times or greater, or both cases.

11. The detection limit calculation device of claim 1, further comprising a control unit that controls the image capture unit, that accumulates charge according to the radiation irradiated and that generates a radiographic image based on accumulated charges, by controlling such that charge accumulation is performed irrespective of a detection result of the detection unit.

12. The detection limit calculation device of claim 11, further comprising a notification unit that notifies that the control unit is controlling such that charge accumulation is performed in the image capture unit.

13. The detection limit calculation device of claim 1, wherein:
the image capture unit comprises a radiation detection device that contains a plurality of pixels, each including respective sensor portions that generate charges according to a radiation amount of irradiated radiation and respective switching elements that read charges from the sensor portions and output to signal lines electrical signals that accord with the charges, and common electrode lines that supply a bias voltage to the sensor portions; and
the detection unit detects that irradiation of radiation has started in cases in which an electrical signal that arises from charges generated in the sensor portion and that flows in the common electrode line satisfies a specific irradiation detection condition.

14. A radiation detection device comprising:
a detection unit that, for image capture of a radiographic image of an imaging subject with an image capture unit, detects whether irradiation of radiation has started based on a radiation amount of radiation comprising radiation that has been irradiated and passed through the imaging subject; and
the detection limit calculation device of claim 1 that calculates the detection limit of the detection unit.

15. A radiographic image capture system comprising:
a detection unit that, for image capture of a radiographic image of an imaging subject with an image capture unit, detects whether irradiation of radiation has started based on a radiation amount of radiation comprising radiation that has been irradiated and passed through the imaging subject;
the detection limit calculation device of claim 1 that calculates a detection limit of the detection unit; and
a control device that controls the image capture unit.

16. A radiographic image capture system comprising:
a radiographic image capture apparatus that comprises a detection unit that detects whether irradiation of radiation has started, and an image capture unit that captures a radiographic image of an imaging subject according to irradiated radiation based on a detection result of the detection unit; and
the detection limit calculation device of claim 1 that calculates a detection limit of the detection unit.

17. A radiographic image capture system comprising:
an irradiation device that irradiates radiation;
a radiographic image capture apparatus that comprises a detection unit that detects whether irradiation of radiation by the irradiation device has started, and an image capture unit that captures a radiographic image of an imaging subject according to irradiated radiation based on a detection result of a detection unit; and
the detection limit calculation device of claim 1 that calculates the detection limit of the detection unit.

18. A non-volatile computer-readable storage medium stored with a detection limit calculation program that causes a computer to:
calculate, for image capture of a radiographic image of an imaging subject with an image capture unit, a detection limit of a detection unit that detects whether irradiation of radiation has started based on a radiation amount of radiation comprising radiation that has been irradiated and passed through the imaging subject, based on imaging subject data relating to the imaging subject or irradiation data relating to irradiation of radiation or both thereof.

19. A detection limit calculation method comprising:
calculating, for image capture of a radiographic image of an imaging subject with an image capture unit, a detection limit of a detection unit that detects whether irradiation of radiation has started based on a radiation amount of radiation comprising radiation that has been irradiated and passed through the imaging subject, based on imaging subject data relating to the imaging subject or irradiation data relating to irradiation of radiation or both thereof.
